SEQUENCE LISTING (13 sequences)

SEQ ID NO: 1 - protein, 1464 aa, Homo sapiens
MFSFVDLRLLLLLAATALLTHGQEEGQVEGQDEDIPPITCVQNGLRYHDRDVWKPEPCRICVCDNGKVLCDDVICDETKNCPGAEVPEGECCPVCPDGSESPTDQETTGVEGPKGDTGPRGPRGPAGPPGRDGIPGQPGLPGPPGPPGPPGPPGLGGNFAPQLSYGYDEKSTGGISVPGPMGPSGPRGLPGPPGAPGPQGFQGPPGEPGEPGASGPMGPRGPPGPPGKNGDDGEAGKPGRPGERGPPGPQGARGLPGTAGLPGMKGHRGFSGLDGAKGDAGPAGPKGEPGSPGENGAPGQMGPRGLPGERGRPGAPGPAGARGNDGATGAAGPPGPTGPAGPPGFPGAVGAKGEAGPQGPRGSEGPQGVRGEPGPPGPAGAAGPAGNPGADGQPGAKGANGAPGIAGAPGFPGARGPSGPQGPGGPPGPKGNSGEPGAPGSKGDTGAKGEPGPVGVQGPPGPAGEEGKRGARGEPGPTGLPGPPGERGGPGSRGFPGADGVAGPKGPAGERGSPGPAGPKGSPGEAGRPGEAGLPGAKGLTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGARGERGFPGERGVQGPPGPAGPRGANGAPGNDGAKGDAGAPGAPGSQGAPGLQGMPGERGAAGLPGPKGDRGDAGPKGADGSPGKDGVRGLTGPIGPPGPAGAPGDKGESGPSGPAGPTGARGAPGDRGEPGPPGPAGFAGPPGADGQPGAKGEPGDAGAKGDAGPPGPAGPAGPPGPIGNVGAPGAKGARGSAGPPGATGFPGAAGRVGPPGPSGNAGPPGPPGPAGKEGGKGPRGETGPAGRPGEVGPPGPPGPAGEKGSPGADGPAGAPGTPGPQGIAGQRGVVGLPGQRGERGFPGLPGPSGEPGKQGPSGASGERGPPGPMGPPGLAGPPGESGREGAPGAEGSPGRDGSPGAKGDRGETGPAGPPGAPGAPGAPGPVGPAGKSGDRGETGPAGPAGPVGPVGARGPAGPQGPRGDKGETGEQGDRGIKGHRGFSGLQGPPGPPGSPGEQGPSGASGPAGPRGPPGSAGAPGKDGLNGLPGPIGPPGPRGRTGDAGPVGPPGPPGPPGPPGPPSAGFDFSFLPQPPQEKAHDGGRYYRADDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 2 - protein, 1366 aa, Homo sapiens [sequence omitted]

SEQ ID NO: 3 - protein, 1464 aa, Homo sapiens; identical to SEQ ID NO: 1

SEQ ID NO: 4 - protein, 1366 aa, Homo sapiens [sequence omitted]

SEQ ID NO: 5 - protein, 1489 aa, Homo sapiens
MAHARVLLLALAVLATAAVAVASSSSFADSNPIRPVTDRAASTLAQLQEEGQVEGQDEDIPPITCVQNGLRYHDRDVWKPEPCRICVCDNGKVLCDDVICDETKNCPGAEVPEGECCPVCPDGSESPTDQETTGVEGPKGDTGPRGPRGPAGPPGRDGIPGQPGLPGPPGPPGPPGPPGLGGNFAPQLSYGYDEKSTGGISVPGPMGPSGPRGLPGPPGAPGPQGFQGPPGEPGEPGASGPMGPRGPPGPPGKNGDDGEAGKPGRPGERGPPGPQGARGLPGTAGLPGMKGHRGFSGLDGAKGDAGPAGPKGEPGSPGENGAPGQMGPRGLPGERGRPGAPGPAGARGNDGATGAAGPPGPTGPAGPPGFPGAVGAKGEAGPQGPRGSEGPQGVRGEPGPPGPAGAAGPAGNPGADGQPGAKGANGAPGIAGAPGFPGARGPSGPQGPGGPPGPKGNSGEPGAPGSKGDTGAKGEPGPVGVQGPPGPAGEEGKRGARGEPGPTGLPGPPGERGGPGSRGFPGADGVAGPKGPAGERGSPGPAGPKGSPGEAGRPGEAGLPGAKGLTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGARGERGFPGERGVQGPPGPAGPRGANGAPGNDGAKGDAGAPGAPGSQGAPGLQGMPGERGAAGLPGPKGDRGDAGPKGADGSPGKDGVRGLTGPIGPPGPAGAPGDKGESGPSGPAGPTGARGAPGDRGEPGPPGPAGFAGPPGADGQPGAKGEPGDAGAKGDAGPPGPAGPAGPPGPIGNVGAPGAKGARGSAGPPGATGFPGAAGRVGPPGPSGNAGPPGPPGPAGKEGGKGPRGETGPAGRPGEVGPPGPPGPAGEKGSPGADGPAGAPGTPGPQGIAGQRGVVGLPGQRGERGFPGLPGPSGEPGKQGPSGASGERGPPGPMGPPGLAGPPGESGREGAPGAEGSPGRDGSPGAKGDRGETGPAGPPGAPGAPGAPGPVGPAGKSGDRGETGPAGPAGPVGPAGARGPAGPQGPRGDKGETGEQGDRGIKGHRGFSGLQGPPGPPGSPGEQGPSGASGPAGPRGPPGSAGAPGKDGLNGLPGPIGPPGPRGRTGDAGPVGPPGPPGPPGPPGPPSAGFDFSFLPQPPQEKAHDGGRYYRADDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLKGSNEIEIRAEGNSRFTYSVTVDGCTSHTGAWGKTVIEYKTTKTSRLPIIDVAPLDVGAPDQEFGFDVGPVCFL

SEQ ID NO: 6 - protein, 1389 aa, Homo sapiens
MAHARVLLLALAVLATAAVAVASSSSFADSNPIRPVTDRAASTLAQLLQEETVRKGPAGDRGPRGERGPPGPPGRDGEDGPTGPPGPPGPPGPPGLGGNFAAQYDGKGVGLGPGPMGLMGPRGPPGAAGAPGPQGFQGPAGEPGEPGQTGPAGARGPAGPPGKAGEDGHPGKPGRPGERGVVGPQGARGFPGTPGLPGFKGIRGHNGLDGLKGQPGAPGVKGEPGAPGENGTPGQTGARGLPGERGRVGAPGPAGARGSDGSVGPVGPAGPIGSAGPPGFPGAPGPKGEIGAVGNAGPTGPAGPRGEVGLPGLSGPVGPPGNPGANGLTGAKGAAGLPGVAGAPGLPGPRGIPGPVGAAGATGARGLVGEPGPAGSKGESGNKGEPGSAGPQGPPGPSGEEGKRGPNGEAGSAGPPGPPGLRGSPGSRGLPGADGRAGVMGPPGSRGASGPAGVRGPNGDAGRPGEPGLMGPRGLPGSPGNIGPAGKEGPVGLPGIDGRPGPIGPAGARGEPGNIGFPGPKGPTGDPGKNGDKGHAGLAGARGAPGPDGNNGAQGPPGPQGVQGGKGEQGPAGPPGFQGLPGPSGPAGEVGKPGERGLHGEFGLPGPAGPRGERGPPGESGAAGPTGPIGSRGPSGPPGPDGNKGEPGVVGAVGTAGPSGPSGLPGERGAAGIPGGKGEKGEPGLRGEIGNPGRDGARGAHGAVGAPGPAGATGDRGEAGAAGPAGPAGPRGSPGERGEVGPAGPNGFAGPAGAAGQPGAKGERGGKGPKGENGVVGPTGPVGAAGPAGPNGPPGPAGSRGDGGPPGMTGFPGAAGRTGPPGPSGISGPPGPPGPAGKEGLRGPRGDQGPVGRTGEVGAVGPPGFAGEKGPSGEAGTAGPPGTPGPQGLLGAPGILGLPGSRGERGLPGVAGAVGEPGPLGIAGPPGARGPPGAVGSPGVNGAPGEAGRDGNPGNDGPPGRDGQPGHKGERGYPGNIGPVGAAGAPGPHGPVGPAGKHGNRGETGPSGPVGPAGAVGPRGPSGPQGIRGDKGEPGEKGPRGLPGFKGHNGLQGLPGIAGHHGDQGAPGSVGPAGPRGPAGPSGPAGKDGRTGHPGTVGPAGIRGPQGHQGPAGPPGPPGPPGPPGVSGGGYDFGYDGDFYRADQPRSAPSLRPKDYEVDATLKSLNNQIETLLTPEGSRKNPARTCRDLRLSHPEWSSGYYWIDPNQGCTMEAIKVYCDFPTGETCIRAQPENIPAKNWYRSSKDKKHVWLGETINAGSQFEYNVEGVTSKEMATQLAFMRLLANYASQNITYHCKNSIAYMDEETGNLKKAVILQGSNDVELVAEGNSRFTYTVLVDGCSKKTNEWGKTIIEYKTNKPSRLPFLDIAPLDIGGADHEFFVDIGPVCFK

SEQ ID NO: 7 - DNA, 5927 bp, Homo sapiens
tcgtcggagcagacgggagtttctcctcggggtcggagcaggaggcacgcggagtgtgaggccacgcatgagcggacgctaaccccctccccagccacaaagagtctacatgtctagggtctagacatgttcagctttgtggacctccggctcctgctcctcttagcggccaccgccctcctgacgcacggccaagaggaaggccaagtcgagggccaagacgaagacatcccaccaatcacctgcgtacagaacggcctcaggtaccatgaccgagacgtgtggaaacccgagccctgccggatctgcgtctgcgacaacggcaaggtgttgtgcgatgacgtgatctgtgacgagaccaagaactgccccggcgccgaagtccccgagggcgagtgctgtcccgtctgccccgacggctcagagtcacccaccgaccaagaaaccaccggcgtcgagggacccaagggagacactggcccccgaggcccaaggggacccgcaggcccccctggccgagatggcatccctggacagcctggacttcccggaccccccggaccccccggacctcccggaccccctggcctcggaggaaactttgctccccagctgtcttatggctatgatgagaaatcaaccggaggaatttccgtgcctggccccatgggtccctctggtcctcgtggtctccctggcccccctggtgcacctggtccccaaggcttccaaggtccccctggtgagcctggcgagcctggagcttcaggtcccatgggtccccgaggtcccccaggtccccctggaaagaatggagatgatggggaagctggaaaacctggtcgtcctggtgagcgtgggcctcctgggcctcagggtgctcgaggattgcccggaacagctggcctccctggaatgaagggacacagaggtttcagtggtttggatggtgccaagggagatgctggtcctgctggtcctaagggtgagcctggcagccctggtgaaaatggagctcctggtcagatgggcccccgtggcctgcctggtgagagaggtcgccctggagcccctggccctgctggtgctcgtggaaatgatggtgctactggtgctgccgggccccctggtcccaccggccccgctggtcctcctggcttccctggtgctgttggtgctaagggtgaagctggtccccaagggccccgaggctctgaaggtccccagggtgtgcgtggtgagcctggcccccctggccctgctggtgctgctggccctgctggaaaccctggtgctgatggacagcctggtgctaaaggtgccaatggtgctcctggtattgctggtgctcctggcttccctggtgcccgaggcccctctggaccccagggccccggcggccctcctggtcccaagggtaacagcggtgaacctggtgctcctggcagcaaaggagacactggtgctaagggagagcctggccctgttggtgttcaaggaccccctggccctgctggagaggaaggaaagcgaggagctcgaggtgaacccggacccactggcctgcccggaccccctggcgagcgtggtggacctggtagccgtggtttccctggcgcagatggtgttgctggtcccaagggtcccgctggtgaacgtggttctcctggccctgctggccccaaaggatctcctggtgaagctggtcgtcccggtgaagctggtctgcctggtgccaagggtctgactggaagccctggcagccctggtcctgatggcaaaactggcccccctggtcccgccggtcaagatggtcgccccggacccccaggcccacctggtgcccgtggtcaggctggtgtgatgggattccctggacctaaaggtgctgctggagagcccggcaaggctggagagcgaggtgttcccggaccccctggcgctgtcggtcctgctggcaaagatggagaggctggagctcagggaccccctggccctgctggtcccgctggcgagagaggtgaacaaggccctgctggctcccccggattccagggtctccctggtcctgctggtcctccaggtgaagcaggcaaacctggtgaacagggtgttcctggagaccttggcgcccctggcccctctggagcaagaggcgagagaggtttccctggcgagcgtggtgtgcaaggtccccctggtcctgctggtccccgaggggccaacggtgctcccggcaacgatggtgctaagggtgatgctggtgcccctggagctcccggtagccagggcgcccctggccttcagggaatgcctggtgaacgtggtgcagctggtcttccagggcctaagggtgacagaggtgatgctggtcccaaaggtgctgatggctctcctggcaaagatggcgtccgtggtctgactggccccattggtcctcctggccctgctggtgcccctggtgacaagggtgaaagtggtcccagcggccctgctggtcccactggagctcgtggtgcccccggagaccgtggtgagcctggtccccccggccctgctggctttgctggcccccctggtgctgacggccaacctggtgctaaaggcgaacctggtgatgctggtgctaaaggcgatgctggtccccctggccctgccggacccgctggaccccctggccccattggtaatgttggtgctcctggagccaaaggtgctcgcggcagcgctggtccccctggtgctactggtttccctggtgctgctggccgagtcggtcctcctggcccctctggaaatgctggaccccctggccctcctggtcctgctggcaaagaaggcggcaaaggtccccgtggtgagactggccctgctggacgtcctggtgaagttggtccccctggtccccctggccctgctggcgagaaaggatcccctggtgctgatggtcctgctggtgctcctggtactcccgggcctcaaggtattgctggacagcgtggtgtggtcggcctgcctggtcagagaggagagagaggcttccctggtcttcctggcccctctggtgaacctggcaaacaaggtccctctggagcaagtggtgaacgtggtccccctggtcccatgggcccccctggattggctggaccccctggtgaatctggacgtgagggggctcctggtgccgaaggttcccctggacgagacggttctcctggcgccaagggtgaccgtggtgagaccggccccgctggaccccctggtgctcctggtgctcctggtgcccctggccccgttggccctgctggcaagagtggtgatcgtggtgagactggtcctgctggtcccgccggtcctgtcggccctgttggcgcccgtggccccgccggaccccaaggcccccgtggtgacaagggtgagacaggcgaacagggcgacagaggcataaagggtcaccgtggcttctctggcctccagggtccccctggccctcctggctctcctggtgaacaaggtccctctggagcctctggtcctgctggtccccgaggtccccctggctctgctggtgctcctggcaaagatggactcaacggtctccctggccccattgggccccctggtcctcgcggtcgcactggtgatgctggtcctgttggtccccccggccctcctggacctcctggtccccctggtcctcccagcgctggtttcgacttcagcttcctgccccagccacctcaagagaaggctcacgatggtggccgctactaccgggctgatgatgccaatgtggttcgtgaccgtgacctcgaggtggacaccaccctcaagagcctgagccagcagatcgagaacatccggagcccagagggcagccgcaagaaccccgcccgcacctgccgtgacctcaagatgtgccactctgactggaagagtggagagtactggattgaccccaaccaaggctgcaacctggatgccatcaaagtcttctgcaacatggagactggtgagacctgcgtgtaccccactcagcccagtgtggcccagaagaactggtacatcagcaagaaccccaaggacaagaggcatgtctggttcggcgagagcatgaccgatggattccagttcgagtatggcggccagggctccgaccctgccgatgtggccatccagctgaccttcctgcgcctgatgtccaccgaggcctcccagaacatcacctaccactgcaagaacagcgtggcctacatggaccagcagactggcaacctcaagaaggccctgctcctccagggctccaacgagatcgagatccgcgccgagggcaacagccgcttcacctacagcgtcactgtcgatggctgcacgagtcacaccggagcctggggcaagacagtgattgaatacaaaaccaccaagacctcccgcctgcccatcatcgatgtggcccccttggacgttggtgccccagaccaggaattcggcttcgacgttggccctgtctgcttcctgtaaactccctccatcccaacctggctccctcccacccaaccaactttccccccaacccggaaacagacaagcaacccaaactgaaccccctcaaaagccaaaaaatgggagacaatttcacatggactttggaaaatatttttttcctttgcattcatctctcaaacttagtttttatctttgaccaaccgaacatgaccaaaaaccaaaagtgcattcaaccttaccaaaaaaaaaaaaaaaaaaagaataaataaataactttttaaaaaaggaagcttggtccacttgcttgaagacccatgcgggggtaagtccctttctgcccgttgggcttatgaaaccccaatgctgccctttctgctcctttctccacaccccccttggggcctcccctccactccttcccaaatctgtctccccagaagacacaggaaacaatgtattgtctgcccagcaatcaaaggcaatgctcaaacacccaagtggcccccaccctcagcccgctcctgcccgcccagcacccccaggccctgggggacctggggttctcagactgccaaagaagccttgccatctggcgctcccatggctcttgcaacatctccccttcgtttttgagggggtcatgccgggggagccaccagcccctcactgggttcggaggagagtcaggaagggccacgacaaagcagaaacatcggatttggggaacgcgtgtcaatcccttgtgccgcagggctgggcgggagagactgttctgttccttgtgtaactgtgttgctgaaagactacctcgttcttgtcttgatgtgtcaccggggcaactgcctgggggcggggatgggggcagggtggaagcggctccccattttataccaaaggtgctacatctatgtgatgggtggggtggggagggaatcactggtgctatagaaattgagatgcccccccaggccagcaaatgttcctttttgttcaaagtctatttttattccttgatatttttctttttttttttttttttttgtggatggggacttgtgaatttttctaaaggtgctatttaacatgggaggagagcgtgtgcggctccagcccagcccgctgctcactttccaccctctctccacctgcctctggcttctcaggcctctgctctccgacctctctcctctgaaaccctcctccacagctgcagcccatcctcccggctccctcctagtctgtcctgcgtcctctgtccccgggtttcagagacaacttcccaaagcacaaagcagtttttccccctaggggtgggaggaagcaaaagactctgtacctattttgtatgtgtataataatttgagatgtttttaattattttgattgctggaataaagcatgtggaaatgacccaaacataa

SEQ ID NO: 8 - DNA, 5411 bp, Homo sapiens
gtgtcccatagtgtttccaaacttggaaagggcgggggagggcgggaggatgcggagggcggaggtatgcagacaacgagtcagagtttccccttgaaagcctcaaaagtgtccacgtcctcaaaaagaatggaaccaatttaagaagccagccccgtggccacgtcccttcccccattcgctccctcctctgcgcccccgcaggctcctcccagctgtggctgcccgggcccccagccccagccctcccattggtggaggcccttttggaggcaccctagggccagggaaacttttgccgtataaatagggcagatccgggctttattattttagcaccacggcagcaggaggtttcggctaagttggaggtactggccacgactgcatgcccgcgcccgccaggtgatacctccgccggtgacccaggggctctgcgacacaaggagtctgcatgtctaagtgctagacatgctcagctttgtggatacgcggactttgttgctgcttgcagtaaccttatgcctagcaacatgccaatctttacaagaggaaactgtaagaaagggcccagccggagatagaggaccacgtggagaaaggggtccaccaggccccccaggcagagatggtgaagatggtcccacaggccctcctggtccacctggtcctcctggcccccctggtctcggtgggaactttgctgctcagtatgatggaaaaggagttggacttggccctggaccaatgggcttaatgggacctagaggcccacctggtgcagctggagccccaggccctcaaggtttccaaggacctgctggtgagcctggtgaacctggtcaaactggtcctgcaggtgctcgtggtccagctggccctcctggcaaggctggtgaagatggtcaccctggaaaacccggacgacctggtgagagaggagttgttggaccacagggtgctcgtggtttccctggaactcctggacttcctggcttcaaaggcattaggggacacaatggtctggatggattgaagggacagcccggtgctcctggtgtgaagggtgaacctggtgcccctggtgaaaatggaactccaggtcaaacaggagcccgtgggcttcctggtgagagaggacgtgttggtgcccctggcccagctggtgcccgtggcagtgatggaagtgtgggtcccgtgggtcctgctggtcccattgggtctgctggccctccaggcttcccaggtgcccctggccccaagggtgaaattggagctgttggtaacgctggtcctgctggtcccgccggtccccgtggtgaagtgggtcttccaggcctctccggccccgttggacctcctggtaatcctggagcaaacggccttactggtgccaagggtgctgctggccttcccggcgttgctggggctcccggcctccctggaccccgcggtattcctggccctgttggtgctgccggtgctactggtgccagaggacttgttggtgagcctggtccagctggctccaaaggagagagcggtaacaagggtgagcccggctctgctgggccccaaggtcctcctggtcccagtggtgaagaaggaaagagaggccctaatggggaagctggatctgccggccctccaggacctcctgggctgagaggtagtcctggttctcgtggtcttcctggagctgatggcagagctggcgtcatgggccctcctggtagtcgtggtgcaagtggccctgctggagtccgaggacctaatggagatgctggtcgccctggggagcctggtctcatgggacccagaggtcttcctggttcccctggaaatatcggccccgctggaaaagaaggtcctgtcggcctccctggcatcgacggcaggcctggcccaattggcccagctggagcaagaggagagcctggcaacattggattccctggacccaaaggccccactggtgatcctggcaaaaacggtgataaaggtcatgctggtcttgctggtgctcggggtgctccaggtcctgatggaaacaatggtgctcagggacctcctggaccacagggtgttcaaggtggaaaaggtgaacagggtccccctggtcctccaggcttccagggtctgcctggcccctcaggtcccgctggtgaagttggcaaaccaggagaaaggggtctccatggtgagtttggtctccctggtcctgctggtccaagaggggaacgcggtcccccaggtgagagtggtgctgccggtcctactggtcctattggaagccgaggtccttctggacccccagggcctgatggaaacaagggtgaacctggtgtggttggtgctgtgggcactgctggtccatctggtcctagtggactcccaggagagaggggtgctgctggcatacctggaggcaagggagaaaagggtgaacctggtctcagaggtgaaattggtaaccctggcagagatggtgctcgtggtgctcctggtgctgtaggtgcccctggtcctgctggagccacaggtgaccggggcgaagctggggctgctggtcctgctggtcctgctggtcctcggggaagccctggtgaacgtggtgaggtcggtcctgctggccccaatggatttgctggtcctgctggtgctgctggtcaacctggtgctaaaggagaaagaggagccaaagggcctaagggtgaaaacggtgttgttggtcccacaggccccgttggagctgctggcccagctggtccaaatggtccccccggtcctgctggaagtcgtggtgatggaggcccccctggtatgactggtttccctggtgctgctggacggactggtcccccaggaccctctggtatttctggccctcctggtccccctggtcctgctgggaaagaagggcttcgtggtcctcgtggtgaccaaggtccagttggccgaactggagaagtaggtgcagttggtccccctggcttcgctggtgagaagggtccctctggagaggctggtactgctggacctcctggcactccaggtcctcagggtcttcttggtgctcctggtattctgggtctccctggctcgagaggtgaacgtggtctaccaggtgttgctggtgctgtgggtgaacctggtcctcttggcattgccggccctcctggggcccgtggtcctcctggtgctgtgggtagtcctggagtcaacggtgctcctggtgaagctggtcgtgatggcaaccctgggaacgatggtcccccaggtcgcgatggtcaacccggacacaagggagagcgcggttaccctggcaatattggtcccgttggtgctgcaggtgcacctggtcctcatggccccgtgggtcctgctggcaaacatggaaaccgtggtgaaactggtccttctggtcctgttggtcctgctggtgctgttggcccaagaggtcctagtggcccacaaggcattcgtggcgataagggagagcccggtgaaaaggggcccagaggtcttcctggcttaaagggacacaatggattgcaaggtctgcctggtatcgctggtcaccatggtgatcaaggtgctcctggctccgtgggtcctgctggtcctaggggccctgctggtccttctggccctgctggaaaagatggtcgcactggacatcctggtacagttggacctgctggcattcgaggccctcagggtcaccaaggccctgctggcccccctggtccccctggccctcctggacctccaggtgtaagcggtggtggttatgactttggttacgatggagacttctacagggctgaccagcctcgctcagcaccttctctcagacccaaggactatgaagttgatgctactctgaagtctctcaacaaccagattgagacccttcttactcctgaaggctctagaaagaacccagctcgcacatgccgtgacttgagactcagccacccagagtggagcagtggttactactggattgaccctaaccaaggatgcactatggatgctatcaaagtatactgtgatttctctactggcgaaacctgtatccgggcccaacctgaaaacatcccagccaagaactggtataggagctccaaggacaagaaacacgtctggctaggagaaactatcaatgctggcagccagtttgaatataatgtagaaggagtgacttccaaggaaatggctacccaacttgccttcatgcgcctgctggccaactatgcctctcagaacatcacctaccactgcaagaacagcattgcatacatggatgaggagactggcaacctgaaaaaggctgtcattctacagggctctaatgatgttgaacttgttgctgagggcaacagcaggttcacttacactgttcttgtagatggctgctctaaaaagacaaatgaatggggaaagacaatcattgaatacaaaacaaataagccatcacgcctgcccttccttgatattgcacctttggacatcggtggtgctgaccaggaattctttgtggacattggcccagtctgtttcaaataaatgaactcaatctaaattaaaaaagaaagaaatttgaaaaaactttctctttgccatttcttcttcttcttttttaactgaaagctgaatccttccatttcttctgcacatctacttgcttaaattgtgggcaaaagagaaaaagaaggattgatcagagcattgtgcaatacagtttcattaactccttcccccgctcccccaaaaatttgaatttttttttcaacactcttacacctgttatggaaaatgtcaacctttgtaagaaaaccaaaataaaaattgaaaaataaaaaccataaacatttgcaccacttgtggcttttgaatatcttccacagagggaagtttaaaacccaaacttccaaaggtttaaactacctcaaaacactttcccatgagtgtgatccacattgttaggtgctgacctagacagagatgaactgaggtccttgttttgttttgttcataatacaaaggtgctaattaatagtatttcagatacttgaagaatgttgatggtgctagaagaatttgagaagaaatactcctgtattgagttgtatcgtgtggtgtattttttaaaaaatttgatttagcattcatattttccatcttattcccaattaaaagtatgcagattatttgcccaaatcttcttcagattcagcatttgttctttgccagtctcattttcatcttcttccatggttccacagaagctttgtttcttgggcaagcagaaaaattaaattgtacctattttgtatatgtgagatgtttaaataaattgtgaaaaaaatgaaataaagcatgtttggttttccaaaagaacatat

SEQ ID NO: 9 - DNA, 4467 bp, Homo sapiens
atggctcacgctcgtgttctcctcctcgctctcgctgttttggcaacagctgctgtggctgtggcttctagttcttcttttgctgattcaaaccctattagacctgttactgatagagcagcttccactttggctcaattgcaagaggagggccaggttgagggccaagatgaggatatccctccaattacatgcgtgcaaaatggcttgcgttaccacgatagggatgtgtggaaacctgaaccttgtcgtatctgtgtgtgtgataacggcaaggtgctctgcgatgatgttatctgcgatgagacaaaaaattgccctggcgctgaagttcctgagggcgagtgttgccctgtgtgccctgatggttccgagtccccaactgatcaggaaactactggcgtggagggcccaaaaggagatactggtccacgtggtcctaggggtccagcaggtcctccaggtagagatggtattccaggccagcctggattgccaggaccaccaggcccacctggcccaccaggacctcctggtcttggtggaaatttcgctccacaactctcttatggctatgatgagaagtcaacaggtggtatttccgttccaggtcctatgggaccatccggaccaagaggtctcccaggtcctccaggtgctcctggacctcaaggctttcaaggacctccaggcgaaccaggagaaccaggcgcttctggaccaatgggcccaaggggaccacctggcccaccaggaaaaaatggcgatgatggcgaagctggaaagcctggtcgtcctggagagagaggtcctcctggcccacagggtgcaagaggcttgccaggaactgctggcttgcctggaatgaagggacataggggcttctccggcctcgatggcgctaagggtgatgctggccctgctggaccaaagggcgagccaggttcccctggagaaaacggtgctcctggacaaatgggtcctcgtggacttccaggagaaaggggtcgtccaggcgctccaggaccagcaggtgctaggggaaacgatggtgcaacaggcgctgctggccctcctggcccaactggtcctgctggccctccaggattcccaggcgcagttggagctaaaggagaagcaggaccacagggccctaggggttctgaaggacctcagggtgttagaggtgaaccaggtcctccaggcccagctggagcagctggtccagcaggaaatccaggtgctgatggtcaacctggagctaagggcgctaatggcgcaccaggtatcgcaggcgcaccaggttttcctggcgctagaggcccaagtggtcctcaaggaccaggtggaccaccaggtccaaaaggcaattctggcgaacctggcgctccaggttctaaaggagatactggtgctaaaggcgaaccaggacctgttggtgttcagggtcctcctggtcctgctggagaagaaggaaaaagaggtgctcgtggagaaccaggaccaactggacttcctggacctcctggtgaacgtggcggacctggctcaaggggtttccctggagctgatggagtggcaggtccaaaaggccctgctggagagagaggttcaccaggtccagctggtcctaagggctcccctggtgaagcaggtagaccaggcgaagcaggattgccaggcgcaaagggattgacaggctctcctggtagtcctggcccagatggaaaaacaggcccaccaggtccagcaggacaagatggacgtccaggcccaccaggtcctcctggagcaaggggacaagctggcgttatgggttttccaggacctaaaggtgctgctggagagccaggaaaggcaggtgaaagaggagttcctggtccaccaggagcagtgggtcctgctggcaaagatggtgaagctggagcacagggccctccaggccctgctggcccagctggcgaacgtggagaacaaggcccagctggtagtccaggatttcaaggattgcctggccctgctggccctccaggagaagcaggaaaacctggagaacaaggagttcctggtgatttgggagcacctggaccttcaggagcacgtggtgaaagaggcttccctggcgagaggggtgttcaaggtccaccaggtccagcaggacctagaggtgctaatggcgctcctggcaacgatggagcaaaaggtgatgctggtgctcctggcgcacctggaagtcagggtgctcctggattgcaaggaatgcctggagagaggggtgctgctggcttgccaggcccaaagggcgataggggtgatgctggaccaaaaggtgctgatggatccccaggaaaagatggagttcgtggtcttactggcccaatcggacctccaggccctgctggcgctccaggtgataagggcgaaagtggcccaagtggacctgctggacctactggtgctagaggtgcacctggtgataggggtgaacctggaccacctggtccagctggttttgctggtcctcctggagctgatggacaacctggcgcaaagggtgaaccaggtgatgctggcgcaaagggagatgctggtccacctggacctgctggtccagcaggcccccctgggccaatcggtaatgttggagcaccaggtgctaagggagctaggggttccgctggtccacctggagcaacaggatttccaggcgctgctggtagagttggcccaccaggcccatccggaaacgcaggccctcctggtcctccaggtcctgctggcaaggagggtggcaaaggaccaaggggcgaaactggccctgctggtagacctggcgaagttggccctcctggaccaccaggtccagcaggagaaaaaggttccccaggagctgatggcccagctggtgctccaggaactccaggccctcaaggtattgctggacagagaggcgttgtgggactccctggtcaaaggggagagagaggatttccaggcttgccaggacctagtggagaacctggaaaacaaggcccatcaggcgctagtggagagcgtggacctcctggccctatgggacctcctggattggctggcccacctggcgaatcaggtcgtgaaggcgcaccaggcgcagaaggatcacctggaagagatggatcccctggtgctaaaggcgatcgtggagaaactggtccagcaggcccaccaggcgcaccaggtgcacctggcgctccaggacctgtgggaccagctggaaaatccggagataggggcgagacaggcccagcaggaccagctggacctgttggccctgctggcgctcgtggaccagcaggacctcaaggaccaaggggagataagggagaaacaggcgaacaaggcgataggggcattaagggtcataggggttttagtggcctccagggtcctcctggcccacctggatcaccaggagaacagggaccatctggtgcttccggcccagctggtccaagaggacctccaggatcagctggtgcacctggaaaagatggtcttaacggtctcccaggaccaatcggccctccaggacctagaggaagaacaggagatgctggccctgttggccctccaggacctcctggtccaccaggtccacctggtcctccatcagctggattcgatttttcatttcttccacagccaccacaagagaaagctcacgatggcggcagatattaccgtgctgatgatgctaacgttgttagggatagagatttggaagtggatacaactttgaaatccctctcccagcaaattgaaaacattagatctccagaaggttcacgtaaaaacccagctagaacatgtcgtgatttgaaaatgtgtcactccgattggaaaagtggtgaatactggattgatccaaatcagggctgtaatctcgatgctatcaaagttttctgtaacatggaaacaggcgaaacatgcgtttatcctactcaaccttccgtggctcagaaaaattggtacatctcaaaaaatcctaaagataagaggcacgtttggttcggtgaaagtatgactgatggatttcaatttgagtacggcggtcaaggtagtgatccagctgatgtggctattcaactcacatttttgcgtcttatgtccacagaggcatcacaaaacatcacttaccactgcaaaaacagtgtggcttatatggatcaacaaacaggaaaccttaagaaggctcttcttttgaagggctcaaacgagattgagattagagcagagggcaactcaaggtttacttattcagttactgttgatggctgcacttcacatactggcgcttggggtaaaacagttatcgagtataagactacaaaaacatcaagactcccaatcattgatgttgctcctctcgatgttggcgctcctgatcaagagttcggttttgatgtgggcccagtttgtttcctc

SEQ ID NO: 10 - DNA, 4167 bp, Homo sapiens
atggctcacgctcgtgttctcctcctcgctctcgctgttttggcaacagctgctgtggctgtggcttcaagttctagttttgctgattccaacccaattcgtccagttactgatagagcagcttccactttggctcaattgcttcaagaagaaactgtgaggaagggccctgctggcgataggggccctaggggcgaaaggggtccaccaggacctccaggcagggatggcgaagatggtccaactggccctcctggacctcctggccctccagggccacccggcttgggcggaaacttcgcagctcaatacgatggcaagggtgttggtcttggtcctggtcctatgggcttgatgggacctagaggcccacctggtgctgctggtgctcctggaccacagggttttcagggaccagctggcgagccaggagagccaggccaaacaggaccagctggtgcaaggggacctgctggacctcctggaaaagctggtgaagatggtcacccaggcaaaccaggacgtcctggcgaaagaggtgttgttggaccacaaggcgctaggggatttccaggtacacctggattgccaggttttaagggcattcgtggtcataacggcctcgatggattgaagggacagcctggcgcacctggcgttaagggtgaacctggagcaccaggtgaaaacggtactcctggccagactggtgcaagaggactcccaggtgaaaggggtagagttggtgctcctggacctgctggagctaggggtagtgatggtagtgttggtcctgtgggccctgctggtccaatcggttccgctggcccacctggattcccaggcgctccaggacctaaaggagaaatcggtgctgtgggtaacgcaggtcctactggtccagcaggtcctcgtggagaagtgggattgccaggactttctggtccagtgggccctccaggcaaccctggagctaacggcttgacaggagctaaaggcgcagcaggactccctggagtggctggcgcaccaggattgcctggtccaaggggtatcccaggccctgttggcgcagctggagctactggtgcacgtggacttgttggcgaaccaggccctgctggatcaaaaggcgagtctggaaataagggagaacctggttctgctggacctcaaggtcctcctggaccttctggagaagaaggaaaaaggggaccaaatggcgaggctggatcagcaggtccaccaggaccacctggacttcgtggatcccctggtagtagaggacttccaggcgctgatggtagagcaggcgttatgggaccaccaggaagtagaggagcatccggtccagcaggagttaggggtcctaacggagatgctggtagaccaggtgaaccaggtcttatgggcccaaggggcctcccaggtagtccaggaaatatcggccctgctggaaaagaaggccctgttggacttccaggtattgatggacgtcctggccctattggcccagcaggtgcaagaggagaacctggcaatattggatttccaggaccaaagggtccaacaggcgatcctggaaaaaatggagataagggtcatgctggattggcaggcgcaaggggcgctcctggtccagatggaaacaacggcgcacagggtccacctggccctcagggtgttcaaggcggaaaaggcgaacaaggcccagctggaccaccaggctttcaaggcttgccaggaccaagtggtccagcaggtgaagttggcaagccaggcgagcgtggacttcatggcgagtttggactccctggaccagcaggaccaaggggtgaaagaggccctcctggagagagtggcgctgctggaccaacaggcccaatcggtagtagaggtcctagtggacctccaggcccagatggaaataagggtgaaccaggagttgtgggcgctgttggaacagctggtccttcaggaccatcaggactcccaggcgagagaggcgctgctggcattcctggaggaaaaggtgaaaaaggcgaacctggcctccgtggcgaaatcggaaatcctggacgtgatggtgctcgtggtgcacacggcgctgtgggcgctccaggccctgctggtgctactggtgatagaggagaggctggcgcagctggcccagcaggtcctgctggcccaaggggtagtcctggtgaaagaggcgaagttggacctgctggccctaacggctttgctggccctgctggagcagcaggtcaacctggcgctaaaggtgaaaggggcggaaagggcccaaaaggtgaaaatggcgttgtgggaccaactggtccagtgggcgcagctggacctgctggtccaaatggaccaccaggaccagcaggtagtagaggagatggtggacctccaggaatgacaggttttccaggtgctgctggtagaacaggacctcctggtcctagtggtatttctggtccaccaggaccaccaggtcctgctggaaaagaaggattgaggggtccacgtggtgatcaaggaccagtgggcagaactggtgaagttggcgcagtgggaccacctggttttgctggagaaaagggcccttctggagaggcaggaacagctggtcctcctggtacacctggacctcaaggacttttgggtgcacctggtattctcggattgccaggaagtaggggcgaacgtggacttcctggcgtggcaggagcagttggagaacctggccctctcggaatcgcaggcccaccaggcgcaagaggaccaccaggagctgttggatcaccaggcgtgaatggtgcacctggcgaggctggtcgtgatggaaacccaggaaatgatggcccaccaggaagagatggtcaacctggacacaaaggcgagaggggctacccaggaaatattggcccagttggtgctgctggcgcaccaggcccacacggtccagttggaccagcaggaaaacacggtaatcgtggcgaaacaggcccttcaggcccagtgggacctgctggtgctgttggcccaagaggaccatctggacctcaaggcattagaggcgataagggagagcctggcgaaaaaggacctagaggcttgcctggttttaaaggacacaacggtctccaaggacttccaggtatcgctggtcatcatggagatcagggtgctcctggatcagtgggtccagcaggtcctagaggcccagcaggcccttccggtccagcaggaaaggatggacgtactggccaccctggaactgtgggccctgctggaattagaggtcctcaaggtcatcagggccctgctggccctccaggtccaccaggtcctccaggcccaccaggagtttcaggtggtggttacgattttggttacgatggtgatttttaccgtgctgatcaacctagaagtgctccttctctccgtcctaaagattatgaagttgatgctactttgaaatcacttaacaaccagattgagactcttctcacacctgagggatcaagaaagaatccagcacgtacatgccgtgatctcagacttagtcacccagagtggtcaagtggctattattggattgatcctaatcagggttgtacaatggaggctatcaaagtttactgtgattttccaactggagagacatgtattagggcacaacctgagaacattccagctaaaaattggtatcgttcctctaaagataagaaacatgtttggctcggagagactattaacgctggttctcagttcgagtataatgttgagggcgttacttctaaagagatggcaactcagctcgcttttatgagattgctcgctaactacgcatcccaaaacatcacttatcactgcaaaaattccattgcatatatggatgaggagacaggaaatttgaagaaagcagttattctccaaggtagtaacgatgttgagcttgtggctgagggaaatagtagattcacttacacagttttggtggatggatgctcaaagaaaactaatgagtggggcaagacaatcattgagtacaagacaaataagccttctaggctcccatttctcgatattgcacctcttgatatcggaggagctgatcacgagttttttgttgatatcggacctgtttgttttaag

SEQ ID NO: 11 - DNA, 1633 bp, Artificial sequence
  Synthetic sequence containing the coding regions of the vascular signal sequence of barley gene for Thiol protease aleurain precursor fused to the human Prolyl 4-hydroxylase beta subunit and flanking regions
ctcgagtaaaccatggctcatgctagggttttgcttttggctcttgctgttcttgctactgctgctgttgctgtggcttcttcttcatctttcgctgattctaacccaattaggccagtgactgatagagctgcttctactcttgctcaattggtcgacatggatgctccagaagaggaggatcacgttcttgtgcttaggaagtctaacttcgctgaagctcttgctgctcacaagtaccttcttgtggagttttatgctccttggtgcggacattgcaaagctcttgctccagagtatgctaaggctgctggaaagttgaaggctgagggatctgaaattaggcttgctaaagtggatgctactgaggagtctgatcttgctcaacagtacggagttaggggatacccaactattaagttcttcaggaacggagatactgcttctccaaaggagtatactgctggaagggaggctgatgatattgtgaactggcttaagaagagaactggaccagctgctactactcttccagatggagctgctgctgaatctcttgtggagtcatctgaggtggcagtgattggattcttcaaggatgtggagtctgattctgctaagcagttccttcaagctgctgaggctattgatgatattccattcggaattacttctaactctgatgtgttctctaagtaccagcttgataaggatggagtggtgcttttcaagaaattcgatgagggaaggaacaatttcgagggagaggtgacaaaggagaaccttcttgatttcattaagcacaaccagcttccacttgtgattgagttcactgagcagactgctccaaagattttcggaggagagattaagactcacattcttcttttccttccaaagtctgtgtctgattacgatggaaagttgtctaacttcaagactgctgctgagtctttcaagggaaagattcttttcattttcattgattctgatcacactgataaccagaggattcttgagttcttcggacttaagaaggaagagtgcccagctgttaggcttattactcttgaggaggagatgactaagtacaagccagagtctgaagaacttactgctgagaggattactgagttctgccacagattccttgagggaaagattaagccacaccttatgtctcaagagcttccagaggattgggataagcagccagttaaggtgttggtgggtaaaaacttcgaggatgtggctttcgatgagaagaagaacgtgttcgtggagttctacgcaccttggtgtggtcactgtaagcagcttgctccaatttgggataagttgggagagacttacaaggatcacgagaacattgtgattgctaagatggattctactgctaacgaggtggaggctgttaaggttcactctttcccaactttgaagttcttcccagcttctgctgataggactgtgattgattacaacggagaaaggactcttgatggattcaagaagttccttgagtctggaggacaagatggagctggagatgatgatgatcttgaggatttggaagaagctgaggagccagatatggaggaggatgatgatcagaaggctgtgtgatgagctc

SEQ ID NO: 12 - DNA, 1723 bp, Artificial sequence
  Synthetic sequence containing the coding regions of the vascular signal sequence of barley gene for Thiol protease aleurain precursor fused to the human Prolyl 4-hydroxylase alpha-1 subunit and flanking regions
ctcgagtaaaccatggctcatgctagggttttgcttttggctcttgctgttcttgctactgctgctgttgctgtggcttcttcttcatctttcgctgattctaacccaattaggccagtgactgatagagctgcttctactcttgctcaattggtcgacatgcacccaggattcttcacttctattggacagatgactgatcttattcacactgagaaggatcttgtgacttctcttaaggattacattaaggctgaggaggataagttggagcagattaagaagtgggctgagaagttggataggcttacttctactgctacaaaagatccagagggattcgttggtcatccagtgaacgctttcaagttgatgaagaggcttaacactgagtggagtgagcttgagaaccttgtgcttaaggatatgtctgatggattcatttctaaccttactattcagaggcagtacttcccaaatgatgaggatcaagtgggagctgctaaggctcttcttaggcttcaggatacttacaaccttgatactgatacaatttctaagggaaaccttccaggagttaagcacaagtctttccttactgctgaggattgcttcgagcttggaaaggttgcatacactgaggctgattactaccacactgagctttggatggaacaagctcttaggcaacttgatgagggagagatttctactattgataaggtgtcagtgcttgattacctttcttacgctgtgtaccagcagggtgatcttgataaggctcttttgcttactaagaagttgcttgagcttgatccagaacatcagagggctaacggaaaccttaagtacttcgagtacattatggctaaggaaaaggatgtgaacaagtctgcttctgatgatcagtctgatcaaaagactactccaaagaagaagggagtggctgttgattatcttcctgagaggcagaagtatgagatgttgtgtaggggagagggtattaagatgactccaaggaggcagaagaagttgttctgcaggtatcacgatggaaacaggaacccaaagttcattcttgctccagctaagcaagaagatgagtgggataagccaaggattattaggttccacgatattatttctgatgctgagattgagattgtgaaggatcttgctaagccaagacttaggagggctactatttctaaccctattactggtgatcttgagactgtgcactacaggatttctaagtctgcttggctttctggatacgagaacccagtggtgtctaggattaacatgaggattcaggatcttactggacttgatgtgtctactgctgaggagcttcaagttgctaactacggagttggaggacaatatgagccacacttcgatttcgctaggaaggatgagccagatgcttttaaggagcttggaactggaaacaggattgctacttggcttttctacatgtctgatgtttctgctggaggagctactgttttcccagaagtgggagcttctgtttggccaaagaagggaactgctgtgttctggtacaaccttttcgcttctggagagggagattactctactaggcatgctgcttgcccagttcttgttggaaacaagtgggtgtcaaacaagtggcttcatgagaggggacaagagtttagaaggccatgcactctttctgagcttgagtgatgagctc

SEQ ID NO: 13 - DNA, 2888 bp, Artificial sequence
  Synthetic sequence containing the coding regions of the vascular signal sequence of barley gene for Thiol protease aleurain precursor fused to the human Lysyl hydroxylase 3 and flanking regions
gcgaattcgctagctatcactgaaaagacagcaagacaatggtgtctcgatgcaccagaaccacatctttgcagcagatgtgaagcagccagagtggtccacaagacgcactcagaaaaggcatcttctaccgacacagaaaaagacaaccacagctcatcatccaacatgtagactgtcgttatgcgtcggctgaagataagactgaccccaggccagcactaaagaagaaataatgcaagtggtcctagctccactttagctttaataattatgtttcattattattctctgcttttgctctctatataaagagcttgtattttcatttgaaggcagaggcgaacacacacacagaacctccctgcttacaaaccagatcttaaaccatggctcacgctagggttttgcttcttgctcttgctgttcttgctactgctgctgttgctgtggcttcttcaagttctttcgctgattctaacccaattaggccagtgactgatagagctgcttctactcttgctcaattgagatctatgtctgatagaccaaggggaagggatccagttaatccagagaagttgcttgtgattactgtggctactgctgagactgaaggataccttagattccttaggagtgctgagttcttcaactacactgtgaggactcttggacttggagaagaatggaggggaggagatgttgctagaactgttggaggaggacagaaagtgagatggcttaagaaagagatggagaagtacgctgatagggaggatatgattattatgttcgtggattcttacgatgtgattcttgctggatctccaactgagcttttgaagaaattcgttcagtctggatctaggcttcttttctctgctgagtctttttgttggccagaatggggacttgctgagcaatatccagaagtgggaactggaaagagattccttaactctggaggattcattggattcgctactactattcaccagattgtgaggcagtggaagtacaaggatgacgatgatgatcagcttttctacactaggctttaccttgatccaggacttagggagaagttgtctcttaaccttgatcacaagtctaggattttccagaaccttaacggtgctcttgatgaggttgtgcttaagttcgataggaacagagtgaggattaggaacgtggcttacgatactcttcctattgtggtgcatggaaacggaccaacaaaactccagcttaactaccttggaaactacgttccaaacggatggactccagaaggaggatgtggattctgcaatcaggataggagaactcttccaggaggacaaccaccaccaagagttttccttgctgtgttcgttgaacagccaactccattccttccaagattccttcagaggcttcttcttttggattacccaccagatagggtgacacttttccttcacaacaacgaggttttccacgagccacacattgctgattcttggccacagcttcaggatcatttctctgctgtgaagttggttggtccagaagaagctctttctccaggagaagctagggatatggctatggatttgtgcaggcaggatccagagtgcgagttctacttctctcttgatgctgatgctgtgcttactaaccttcagactcttaggattcttattgaggagaacaggaaagtgattgctccaatgctttctaggcacggaaagttgtggtctaatttctggggtgctctttctcctgatgagtactacgctagatcagaggactacgtggagcttgttcagagaaagagagtgggagtttggaacgttccttatatttctcaggcttacgtgattaggggagatactcttaggatggagcttccacagagggatgttttctctggatctgatactgatccagatatggctttctgcaagtctttcagggataagggaattttccttcacctttctaaccagcatgagttcggaagattgcttgctacttcaagatacgatactgagcaccttcatcctgatctttggcagattttcgataacccagtggattggaaggagcagtacattcacgagaactactctagggctcttgaaggagaaggaattgtggagcaaccatgcccagatgtttactggttcccacttctttctgagcaaatgtgcgatgagcttgttgctgagatggagcattacggacaatggagtggaggtagacatgaggattctaggcttgctggaggatacgagaacgttccaactgtggatattcacatgaagcaagtgggatacgaggatcaatggcttcagcttcttaggacttatgtgggaccaatgactgagtctcttttcccaggataccacactaaggctagggctgttatgaacttcgttgtgaggtatcgtccagatgagcaaccatctcttaggccacaccacgattcttctactttcactcttaacgtggctcttaaccacaagggacttgattatgagggaggaggatgccgtttccttagatacgattgcgtgatttcttcaccaagaaagggatgggctcttcttcatccaggaaggcttactcattaccacgagggacttccaactacttggggaactagatatattatggtgtctttcgtggatccatgactgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtccagaattcgcg

(19) United States
(12) Patent Application Publication
(10) Pub. No.: US 2013/0230573 A1
(43) Pub. Date: Sep. 5, 2013

(54) COLLAGEN STRUCTURES AND METHOD OF FABRICATING THE SAME

(71) Applicants: Oded Shoseyov, Karmei Yosef (IL); Amit Yaari, Kibbutz Ein Dror (IL)

(72) Inventors: Oded Shoseyov, Karmei Yosef (IL); Amit Yaari, Kibbutz Ein Dror (IL)

(21) Appl. No.: 13/885,690

(22) PCT Filed: Nov. 16, 2011
(86) PCT No.: PCT/IL11/00886

§ 371 Date: May 16, 2013

Related U.S. Application Data
(60) Provisional application No. 61/414,032, filed on Nov. 16, 2010.
(60) Provisional application No. 61/487,741, filed on May 19, 2011.

(30) Foreign Application Priority Data
Nov. 24, 2010 (IL) PCT/IL2010/000984

(51) Int. Cl. A61K 38/39

(52) U.S. Cl. CPC A61K 38/39
USPC 424/400; 514/17.2; 514/16.7; 514/17.1

(57) ABSTRACT

A method of fabricating a collagen structure, is disclosed. The method comprises applying onto a surface in a layerwise manner acidic solutions of liquid crystalline collagen and a crosslinker to form a layered structure. The acidic solutions that correspond to at least two adjacent layers have different concentrations of the crosslinker therein.

### RELATED APPLICATIONS

This application claims the benefit of priority from U.S. Patent Application No. 61/414,032, filed Nov. 16, 2010, International Patent Application No. PCT/IL2010/000984 filed Nov. 24, 2010, and U.S. Patent Application No. 61/487,741, filed May 19, 2011, the contents of which are hereby incorporated by reference as if fully set forth herein.

### FIELD AND BACKGROUND OF THE INVENTION

The present invention, in some embodiments thereof, relates to non-woven structures and, more particularly, but not exclusively, to collagen structures and method suitable for fabricating collagen structures.

Collagen is the principal structural protein in the body and constitutes approximately one-third of the total body protein. It comprises most of the organic matter of the skin, tendons, bones and teeth and occurs as fibrous inclusions in most other body structures. Some of the properties of collagen are its high tensile strength; its ion exchanging ability, due in part to the binding of electrolytes, metabolites and drugs; its low antigenicity, due to masking of potential antigenic determinants by the helical structure, and its low extensibility, semipermeability, and solubility.

Furthermore collagen is a natural substance for cell adhesion. These properties make this protein suitable for fabrication of bioremodelable research products and medical devices such as implantable prostheses, cell growth substrates, and cellular and acellular tissue constructs.

Naturally, collagen is secreted by cells as a long triple-helical monomer, which polymerizes spontaneously into fibrils and strands, which often have a preferential orientation essential to the function of tissues such as skin, bone and nerve.

The exact structure of the collagen fibril is still unknown, but increasingly detailed models are becoming available, emphasizing the relation between fibril structure and function. Current models hint at a semi-crystalline (liquid crystal like) structure, combining a highly ordered arrangement in the axial direction and a short-range liquid-like order in the lateral direction.

Collagen in its monomeric form is soluble in cold acidic pH (˜pH 2) solutions, and can be precipitated in the form of fibrils by neutralizing the pH, increasing the temperature and/or the ionic strength. Fibrillogenesis is entropy driven. The loss of water molecules from monomer surfaces drives the collagen monomers out of solution and into assemblies with a circular cross-section so as to minimize surface area.

The fibrils formed in-vitro display D-banding pattern of 67 nm wide cross striations typical of natural collagen fibrils formed in-vivo, but lack altogether the macroscopic order that is the basis of structural tissues. Fibrils precipitated out of bulk solutions form an entangled mesh reminiscent of spaghetti and not the neatly ordered arrays of fibrils observed in nature.

Collagen can be deposited from solution by a variety of processes including casting, lyophilization, electrospinning and other processes well known to one skilled in the art. In most of these procedures, collagen fibers of widely varying diameters and lengths from the micrometer range typical of conventional fibers down to the nanometer range are formed. Owing to their small diameters, electrospun fibers possess very high surface-to-area ratios and are expected to display morphologies and material properties very different from their conventional counterparts occurring in nature.

Numerous attempts to direct or align collagen fibrils for manufacturing of collagen matrices have been performed, employing various methods. Major efforts are aimed at creating 2D (collagen surface) or 3D (collagen scaffold) matrices. Exemplary methods include: alignment by surface templating, chemical patterning, nanolithography, electrochemical fabrication, use of a magnetic field and by shear flow.

Studies have shown that, in vitro, collagen displays mesophase (liquid crystalline) properties at concentrations above 20 mg/ml (depending on acid concentration of the solvent). At concentrations between about 20 and about 50 mg/ml diffuse nematic phases appear in the bulk isotropic solution, observed as birefringent flakes. When the collagen concentration is increased, precholesteric patterns form—observed as spherulites, bands, or zigzag extinction patterns. Further increase in the concentration leads to formation of cholesteric patterns that become more and more compact until the entire sample displays characteristic fingerprint pattern.

At concentrations above 150 mg/ml, collagen fibrillar aggregates start to appear even in acidic solution, displaying the 67 nm banding typical of collagen fibrils, in a process reminiscent of a cholesteric-to-smectic (N*/SmA) transition.

U.S. Pat. No. 7,057,023 teaches spinning of liquid crystalline silk to generate silk fibers.

U.S. Patent Application No. 20070187862 teaches spinning a solution of liquid crystalline silk, wherein the solution is devoid of organic solvents to generate silk fibers.

U.S. Patent Application No. 20090069893 teaches formation of oriented collagen based materials from mesophase collagen by application of a shear force.

U.S. Pat. No. 7,048,963 discloses a method of producing an oriented layer of polymer material, by introducing a shearing flow to a free surface in a predominantly monomeric solution of the self-assembling polymer sub-units, and inducing polymerization or growth of the monomer while in this shearing flow.

### SUMMARY OF THE INVENTION

Some embodiments of the invention concern with a method suitable for fabricating a collagen structure. Acidic solutions of liquid crystalline collagen and a crosslinker are applied onto a surface in a layerwise manner to form a layered structure. The pH of the solutions is sufficiently low (for example, 2-5) so as to maintain the crosslinker in an inactive form. Thus, although the crosslinker is present, the crosslinking is suppressed or, more preferably, completely prevented due to the acidity of the solutions. The concentration of the collagen in the solutions is preferably sufficiently high to display liquid crystalline properties. For example, the solutions can show birefringence under crossed polarizers. Typical concentrations can be from about 3 to about 300 mg/ml, depending on the pH level.

In various exemplary embodiments of the invention acidic solutions that correspond to two or more adjacent layers have different concentrations of the crosslinker therein. This allows different layers to possess different levels of crosslinking hence to encode a predetermined three-dimensional shape into the layered structure and to provide the structure with a self-shapeable property as further detailed hereinunder.

In some embodiments of the present invention each layer is at least partially dried prior to the application of a subsequent layer. Alternatively or additionally, the layered structure can be dried collectively once all the layers are applied.

In the grafting procedure of the present embodiments, the crosslinking groups are covalently crosslinked to the collagen but are not activated, optionally and preferably such that there is no crosslinking occurs between the collagen monomers. Thus, crosslinking groups chemically modify the collagen. When collagen crosslinking is desired, the grafted crosslinking groups are activated (for example, by exerting photoinitiation) and crosslink the collagen monomers together. The level of crosslinking according to some embodiments of the present invention is be determined by the amount or concentration of the grafted groups.

According to an aspect of some embodiments of the present invention there is provided an article, which comprises a plurality of layers each being made of a non-fibrilized collagen material and being incorporated with an activatable crosslinker which is in an inactive form. The layers are arranged to form a layered structure. Optionally and preferably two or more adjacent layers have different concentrations of the crosslinker therein. The article can be formed by applying solutions of liquid crystalline collagen and the crosslinker onto a surface in a layerwise manner as described above.

In some embodiments of the present invention the crosslinker is activated, and the collagen material is fibrilized, preferably contemporaneously. The activation and fibrilization can be in a suitable coagulation medium at a suitable temperature (e.g., 25-38° C.) and for a suitable period of time (e.g., 10-100 hours). The level of crosslinking in each layer depends of the concentration of the crosslinker. Thus, the level of crosslinking varies among different layers (although some layers may possess the same level of crosslinking as the case may be). Thereafter, a thermal treatment can be applied to the article, for example, by immersing it in a suitable buffer or water at elevated temperatures, so as to induce shrinkage. Due to the difference in the level of crosslinking, the amount of shrinkage is not the same for all layers, and the article acquires a three-dimensional shape.

As a representative example, consider an article with two layers as schematically illustrated in FIGS. 2A-B. The structure comprises of a layer A and a layer B. Layer A contains a larger concentration of the crosslinker than layer B. For layer A can contain 0.5% GTA (Glutaraldehyde) and layer B can contain 0.1% GTA. The article can be crosslinked and fibrilized simultaneously by immersion it in a fibrilogenesis buffer. For example, the buffer can contain a buffering agent, salt and 0.1% GTA. The article is then subjected to thermal treatment for inducing shrinkage. FIG. 2A shows the article after incubation in the buffer, and FIG. 2B shows the article after the shrinkage. As shown, the level of shrinkage is higher for layer B than for layer A, resulting in a convex shape in the direction of the GTA gradient.

Generally, the level of shrinkage is controlled a priori by the crosslinking concentration, the crosslinking and fibrillogenesis period and temperature, and by the temperature and time of the thermal treatment. For given crosslinking and fibrillogenesis period and temperature, and given the temperature and time of the thermal treatment, the level of shrinkage of each layer is inversely proportional to its level of crosslinking.

Thus, the present embodiments provide an article which comprises a plurality of layers each being made of a fibrilized and crosslinked collagen material, wherein one or more layers are non-planar and are characterized by a level of crosslinking which is different from a level of crosslinking characterizing another layer.

According to some embodiments of the invention the method wherein the surface is a solid surface.

According to some embodiments of the invention the method wherein the surface is a surface of a liquid.

According to some embodiments of the invention the method wherein the surface is a surface of a gel.

According to some embodiments of the invention the method comprises at least partially drying each layer prior to the application of a subsequent layer.

According to some embodiments of the invention the method comprises drying the layered structure collectively.

According to some embodiments of the invention the acidic solutions is applied generally along the same direction.

According to some embodiments of the invention the acidic solutions is applied generally along the same direction for all layers.

According to some embodiments of the invention the method comprises activating the crosslinker.

According to some embodiments of the invention the method comprises fibrilizing the collagen.

According to some embodiments of the invention the fibrillization and the activation are executed contemporaneously.

According to some embodiments of the invention the fibrillization comprises immersing the article in a coagulation medium.

According to some embodiments of the invention the crosslinker comprises a material selected from the group consisting of Glutaraldehyde, DOPA, Tyramine and Methacryl.

According to an aspect of some embodiments of the present invention there is provided an article, comprising a plurality of layers each being made of a fibrilized and crosslinked collagen material, the layers being arranged to form a layered structure, wherein at least one layer in the layered structure is characterized by a level of crosslinking which is different from a level of crosslinking characterizing a layer being adjacent thereto.

According to some embodiments of the invention a characteristic direction of alignment of the collagen material in the at least one layer is generally the same as a characteristic direction of alignment of the collagen material in the adjacent layer.

According to some embodiments of the invention the article is at least partially shaped as a spiral.

According to some embodiments of the invention the article has at least one void between adjacent layers.

According to some embodiments of the invention the article is adapted for implantation in an organ of a mammal.

According to some embodiments of the invention the article serves as or is incorporated in an artificial organ.

According to some embodiments of the invention the article is adapted for replacing a part of an organ.

According to some embodiments of the invention the article is adapted for patching, coating or wrapping an organ.

According to some embodiments of the invention the article is adapted for connecting organs.

According to some embodiments of the invention the organ is selected from the group consisting of a bladder, a tendon, a bone, a brain, a cartilage, an esophagus, a fallopian tube, a heart valve, a pancreas, intestines, a gallbladder, a kidney, a liver or liver lobule, a lung or an alveolar structure, a skeletal muscle, skin, a spleen, a stomach, a thymus, a thyroid, a trachea, an ureter, a urethra, a urogenital tract, a uterus, a blood vessel and a cornea.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings and images. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a flowchart diagram of a method suitable for fabricating a collagen structure according to various exemplary embodiments of the present invention;

FIGS. 2A-B are schematic illustrations of a collagen layered structure with two layers, according to some embodiments of the present invention;

FIG. 3A-B are Scanning Electron Microscopy (SEM) images of a spirally shaped collagen layered structure, as obtained in experiments performed according to some embodiments of the present invention; and

FIGS. 4A-C are schematic illustration of a procedure for fabricating a hollow collagen structure, according to some embodiments of the present invention.

### DESCRIPTION OF SPECIFIC EMBODIMENTS OF THE INVENTION

The present invention, in some embodiments thereof, relates to non-woven structures and, more particularly, but not exclusively, to collagen structures and method suitable for fabricating collagen structures.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

Collagen matrix in many biological systems has a very highly ordered liquid crystal structure (mesophase). It is this natural state which provides collagen with its long-range orientation.

The highly ordered mesophase state of naturally occurring collagen can be mimicked in vitro by increasing the concentration of a solution of monomeric collagen above about 20 mg/ml (depending on acid concentration of the solvent).

The present inventors propose that preservation of the crystalline order instilled by the mesophase state of collagen following extrusion, would allow for the generation of structures of collagen fibers with a highly organized collagen structure, thereby providing the collagen structure with superior mechanical properties.

The present inventors showed that extruding fibers from mesophase collagen directly into a coagulating solution maintains and preserves the crystalline structure assumed by the collagen in the mesophase.

The term “collagen” as used herein, refers to a polypeptide having a triple helix structure and containing a repeating Gly-X-Y triplet, where X and Y can be any amino acid but are frequently the imino acids proline and hydroxyproline. According to one embodiment, the collagen is a type I, II, III, V, XI, or biologically active fragments therefrom.

The present inventors found that a collagen structure of predetermined shape can be fabricated by controlling the level of crosslinking at least along the thickness direction of a layered structure. It was envisaged by the present inventors that different levels of crosslinking across provides the layers with a controllable curvature which can be selected in accordance with the desired shape of the structure. Consider, for example, two layers wherein the extent of crosslinking is higher for one layer than for an adjacent layer. As a result of the difference in cross linking, the two layers acquire a curvature such that the layer with higher extent of crosslinking becomes convex and the layer with lower extent of crosslinking becomes concave.

While conceiving the present invention it was uncovered that the collagen structure can be fabricated such that shape information is encoded into the collagen structure, but is not manifested. Thus, the collagen structure has a first shape upon manufacturing (e.g., a flat shape), and a second shape following activation. The shape information can be encoded by controlling the concentration of crosslinker in the layers or regions while suppressing the activity of the crosslinker thereby reducing or, more preferably, inhibiting crosslinking process within the structure. The activity of crosslinker can be suppressed using a solution of sufficiently low pH, for example, from about 2 to about 5.

When it is desired to shape the collagen structure according to the encoded shape information, the crosslinker is activated (for example, by exerting photoinitiation) and crosslinking occurs between the collagen monomers.

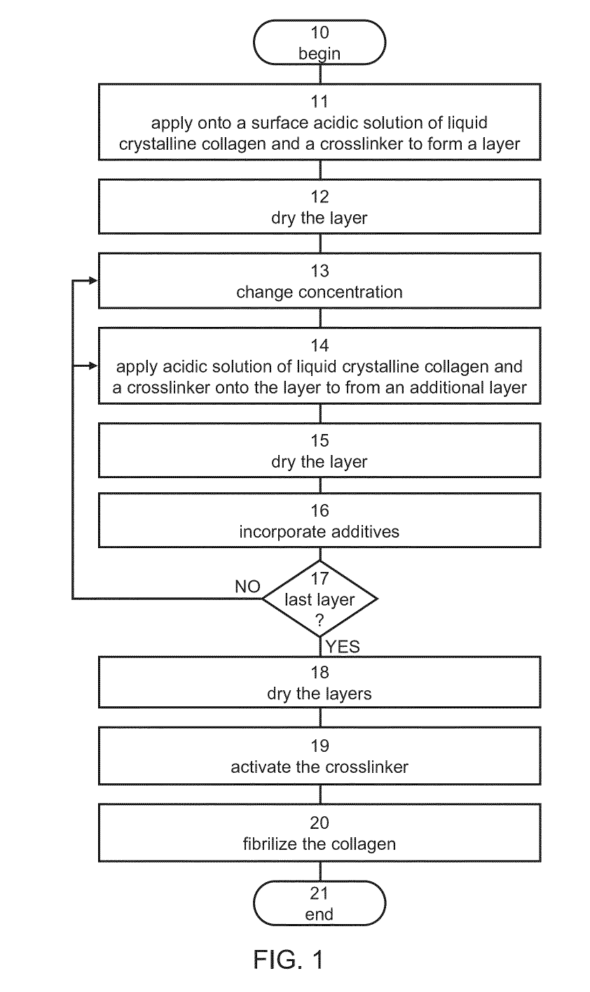

Referring now to the drawings, FIG. 1 is a flowchart diagram of a method suitable for fabricating a collagen structure according to various exemplary embodiments of the present invention. It is to be understood that, unless otherwise defined, the operations described hereinbelow can be executed either contemporaneously or sequentially in many combinations or orders of execution. Specifically, the ordering of the flowchart diagrams is not to be considered as limiting. For example, two or more operations, appearing in the following description or in the flowchart diagrams in a particular order, can be executed in a different order (e.g., a reverse order) or substantially contemporaneously. Additionally, several operations described below are optional and may not be executed.

The method begins at 10 and continues to 11 at which a first layer of acidic solution of liquid crystalline collagen and a crosslinker is applied to a surface. The surface can be of any type, including, without limitation, a solid surface (e.g., a glass surface, a solid polymer, etc.), a surface of a gel (e.g., a biocompatible gel) and a surface of a liquid (e.g., water, oil, etc.). Many types of crosslinkers are contemplated. Representative examples including, without limitation, Glutaraldehyde, DOPA, Tyramine and Methacryl.

In some embodiments of the present invention the method continues to 12 at which the layer is dried, at least partially. The drying can be done by any drying technique, such as, but not limited to, using a stream of gas (e.g., air) or the like. In some embodiments, however, 12 is not executed and the method proceeds without drying the layer.

The method optionally continues to 13 at which the concentration of the crosslinker in the acidic solution is changed, so as to provide a different acidic solution. The method can increase or decrease the concentration, depending on the shape information that is to be encoded into the structure. The method continues to 14 at which a layer of acidic solution of liquid crystalline collagen and a crosslinker is applied to the first layer. From 14 the method optionally continues to 15 at which the layer is dried as further detailed hereinabove. Alternatively the method can proceed without drying the layer.

At 16 one or more additives are optionally and preferably incorporated into the applied layer(s). The additives may include chemical or biological material. For example, the additive may include therapeutic compounds or agents, agents acting to increase cell attachment, cell spreading, cell proliferation, cell differentiation and/or cell migration, cells, culture medium, and the like, as further detailed hereinunder. Although the incorporation 16 of additive(s) is shown in FIG. 1 after operation 15, this need not necessarily be the case since the present inventors contemplate many orders of executions. Thus, the incorporation 16 of additive(s) can be performed individually for one or more of the layer, prior to the application of the next layer, or it can be performed only on the top most or bottom most layers of the collagen structure. The incorporation 16 of additive(s) can be performed either while the layers are still wet, or after the layers are dried (in embodiments in which the drying operation is employed). The incorporation of additive(s) may optionally be performed by mixing the respective additive(s) into the acidic solution prior before applying the solution to form the respective layer.

The method continues to decision 17 at which the method determine whether the most recently applied layer is the last layer to be applied. Namely, at 17 the method compares the number of layer already applied to the desired number of layers.

If the most recently applied layer is not the last layer, the method loops back to optional operation 13 (if it is desired to change the concentration of the crosslinker in the subsequent layer) or directly to 14 (if it is desired to have two adjacent layers with equal concentrations).

Two or more of the layers can be applied along the same horizontal direction. In some embodiments, all layers are applied along the same horizontal direction. Alternatively, some layers can be applied along different directions. For example, one or more layers can be applied along one direction and one or more layers can be applied along another (e.g., orthogonal) direction.

In various exemplary embodiments of the invention the acidic solution applied in at least one of the layers has a characteristic pH is such that the activity of crosslinker is suppressed or inhibited. Typical pH level for such suppression is from about 2 to about 5. These embodiments are particularly useful when it is desired to encode the shape information without actually shaping the structure according to the encoded information. Preferably, all the layers have pH which is from about 2 to about 5.

If the most recently applied layer is the last layer, the method optionally and preferably continues to 18 at which the layers are dried collectively. Operation 18 can be executed, for example, when operations 12 and/or 15 are skipped.

At optional operation 19 the crosslinker is activated. The activation procedure depends on the type of crosslinker which is used, and the skilled person would know how to elect the appropriate technique for a given type of crosslinker. Representative example of activation procedure including, without limitation, photoinitiation, thermal initiation and the like. At optional operation 20 the collagen is fibrilized. Typically, fibrilization is achieved by immersing the layers in a fibrilogenesis buffer coagulation medium. Use of thermal treatment in conjunction with the fibrilogenesis buffer is also contemplated. In various exemplary embodiments of the invention both operations 19 and 20 are executed simultaneously, such that the crosslinking occurs together with the fibrilization. In some embodiments, operations 19 and 20 are not executed and the method ends without activating the crosslinker and fibrilizing the collagen. In these embodiments, the fabricated structure is optionally packed for shipping and/or storage.

The method ends at 21.

The present embodiments provide an article which comprises a plurality of layers, where each layer is made of a non-fibrilized collagen material and being incorporated with an activatable crosslinker which is in an inactive form. The layers are arranged to form a layered structure wherein at least two adjacent layers in have different concentrations of the crosslinker therein. Preferably, the layers bare substantially dry (e.g., have water content of less than 5%).

The advantage of having an article with non-fibrilized collagen and a crosslinker in an inactive form is that it can be stored for a prolonged period of time and be activated and fibrilized only prior to its use.

The present embodiments also provide an article which comprising a plurality of layers, where each layer is made of a fibrilized and crosslinked collagen material. The layers are arranged to form a layered structure, wherein at least one layer is characterized by a level of crosslinking which is different from a level of crosslinking characterizing a layer being adjacent thereto.

The advantage of having an article with fibrilized and crosslinked collagen material is that its encoded shape can be obtained without applying activation and fibrilization. For example, the encoded shape can be obtained by thermal treatment, e.g., by increasing the temperature of the article to a temperature which is above 60° C. or above 70° C. or above 80° C., say 90° C. or more.

Once the shape article of the article of any of the above embodiments is obtained, at least one of its layers preferably acquire a curvature and becomes a non-planar layer. In some embodiments of the present invention the article, once brought to its encoded shape, as one or more voids between adjacent layers thereof. The overall shape of the article can be according to any geometry. Specifically, geometries of zero sectional curvature, geometries of non-zero constant sectional curvature and geometries of non-constant sectional curvature are contemplated.

Representative examples of shapes that the article of the present embodiments can assume, include, without limitation, a cylinder, a spiral, a disk, an oval, a cuboid, a prism, a sphere, a hemisphere, a portion of a sphere, a spheroid, a portion of a spheroid, a prolate spheroid, an oblate spheroid, an ellipsoid, a portion of ellipsoid, a hyperboloid, a portion of a hyperboloid, a paraboloid, a portion of a paraboloid a cylindrical shell, a portion of a cylindrical shell, a polyhedron shell, a portion of a polyhedron shell, and any combination between two or more of these shapes.

In various exemplary embodiments of the invention the article is adapted for implantation in an organ of a mammal. The implantation can be in an open surgery or minimally invasive procedure in which case the article is preferably implantable in an internal organ, or by external implantation. While, being implanted in the body of the mammal, the article can have many uses. In some embodiments the article serves as a scaffold for tissue regeneration, in some embodiments the article replaces a part of an organ, in some embodiments the article serves as an artificial organ, In some embodiments the article serves as a prosthesis, in some embodiments the article serves for patching, coating or wrapping the organ, and in some embodiments, the article connects two organs.

The article of the present embodiments can be shaped so as to fit to replace, or be implanted in, any organ in the body, including, without limitation, the bladder, a tendon, a bone, the brain, a cartilage, the esophagus, the fallopian tube, a heart valve, the pancreas, the intestines, the gallbladder, a kidney, the liver, a liver lobule, a lung, an alveolar structure, a skeletal muscle, a skin part, the spleen, the stomach, the thymus, the thyroid, the trachea, the ureter, the urethra, the urogenital tract, the uterus, a blood vessel and a cornea.

When the article serves as a scaffold, it can have many uses, including, without limitation, as a dermal layer of artificial skin, a dental bone graft substitute, a cartilage defect repair implant, an osteochondral defect repair implant, a spine fusion element, scaffold for the treatment of bone fractures.

As used herein, the term “scaffold” refers to a 3D matrix upon which cells may be cultured (i.e., survive and preferably proliferate for a predetermined time period).

The scaffold may be fully comprised of the collagen structure of the present embodiments or composites thereof, or may comprise a solid support on which the collagen structure is placed.

A “solid support,” as used refers to a three-dimensional matrix or a planar surface (e.g. a cell culture plate) on which cells may be cultured. The solid support can be derived from naturally occurring substances (i.e., protein based) or synthetic substances. Suitable synthetic matrices are described in, e.g., U.S. Pat. Nos. 5,041,138, 5,512,474, and 6,425,222. For example, biodegradable artificial polymers, such as polyglycolic acid, polyorthoester, or polyanhydride can be used for the solid support. Calcium carbonate, aragonite, and porous ceramics (e.g., dense hydroxyapatite ceramic) are also suitable for use in the solid support. Polymers such as polypropylene, polyethylene glycol, and polystyrene can also be used in the solid support.

The scaffold of the present embodiments or a portion thereof can be incorporated with therapeutic compounds or agents that modify cellular activity, preferably ex-vivo. The therapeutic compounds can be attached to, coated on, embedded or impregnated into the scaffold. In addition, agents that act to increase cell attachment, cell spreading, cell proliferation, cell differentiation and/or cell migration in the scaffold may also be incorporated into the scaffold, preferably ex-vivo. Such agents can be biological agents such as an amino acid, peptides, polypeptides, proteins, DNA, RNA, lipids and/or proteoglycans.

Suitable proteins which can be used along with the present embodiments include, but are not limited to, extracellular matrix proteins [e.g., fibrinogen, collagen, fibronectin, vimentin, microtubule-associated protein 1D, Neurite outgrowth factor (NOF), bacterial cellulose (BC), laminin and gelatin], cell adhesion proteins [e.g., integrin, proteoglycan, glycosaminoglycan, laminin, intercellular adhesion molecule (ICAM) 1, N-CAM, cadherin, tenascin, gicerin, RGD peptide and nerve injury induced protein 2 (ninjurin2)], growth factors [epidermal growth factor, transforming growth factor-α, fibroblast growth factor-acidic, bone morphogenic protein, fibroblast growth factor-basic, erythropoietin, thrombopoietin, hepatocyte growth factor, insulin-like growth factor-I, insulin-like growth factor-II, Interferon-β, platelet-derived growth factor, Vascular Endothelial Growth Factor and angiopeptin], cytokines [e.g., M-CSF, IL-1beta, IL-8, beta-thromboglobulin, EMAP-II, G-CSF and IL-10], proteases [pepsin, low specificity chymotrypsin, high specificity chymotrypsin, trypsin, carboxypeptidases, aminopeptidases, proline-endopeptidase, Staphylococcus aureus V8 protease, Proteinase K (PK), aspartic protease, serine proteases, metalloproteases, ADAMTS17, tryptase-gamma, and matriptase-2] and protease substrates.

Additionally and/or alternatively, the scaffold of the present embodiments may comprise an antiproliferative agent (e.g., rapamycin, paclitaxel, tranilast, Atorvastatin and trapidil), an immunosuppressant drug (e.g., sirolimus, tacrolimus and Cyclosporine) and/or a non-thrombogenic or anti-adhesive substance (e.g., tissue plasminogen activator, reteplase, TNK-tPA, glycoprotein IIb/IIIa inhibitors, clopidogrel, aspirin, heparin and low molecular weight heparins such as enoxiparin and dalteparin). Such substances are optionally and preferably incorporated into the article of the present embodiments ex vivo.

In some embodiments of the present invention the article of the present embodiments is seeded with cells ex vivo. Cells which may be seeded on the collagen of the present embodiments may comprise a heterogeneous population of cells or alternatively the cells may comprise a homogeneous population of cells. Such cells can be for example, stem cells (such as embryonic stem cells, bone marrow stem cells, cord blood cells, mesenchymal stem cells, adult tissue stem cells), progenitor cells, or differentiated cells such as chondrocytes, osteoblasts, connective tissue cells (e.g., fibrocytes, fibroblasts and adipose cells), endothelial and epithelial cells. The cells may be naive or genetically modified.

In some embodiments, the cells are mammalian in origin.

The cells may optionally be of autologous origin or non-autologous origin, such as postpartum-derived cells (as described in U.S. application Ser. Nos. 10/887,012 and 10/887,446). Typically, the cells are selected according to the tissue being generated.

Techniques for seeding cells onto or into a scaffold are well known in the art, and include, without being limited to, static seeding, filtration seeding and centrifugation seeding.

In various exemplary embodiments of the invention the cells are seeded on the collagen structure of the present embodiments in the presence of a culture medium, so as to facilitate cell growth.

The culture media suitable used for the present It will be appreciated that to support cell growth comprise any liquid medium which allows at least cell survival. Such a culture medium can include, for example, salts, sugars, amino acids and minerals in the appropriate concentrations and with various additives and those of skills in the art are capable of determining a suitable culture medium to specific cell types. Non-limiting examples of such culture medium include, phosphate buffered saline, DMEM, MEM, RPMI 1640, McCoy's 5A medium, medium 199 and IMDM (available e.g., from Biological Industries, Beth Ha'emek, Israel; Gibco-Invitrogen Corporation products, Grand Island, N.Y., USA).

The culture medium may be supplemented with various antibiotics (e.g., Penicillin and Streptomycin), growth factors or hormones, specific amino acids (e.g., L-glutamin) cytokines and the like.

The scaffold of the present embodiments can be administered to subjects in need thereof for the regeneration of tissue such as connective tissue, muscle tissue such as cardiac tissue and pancreatic tissue. Examples of connective tissues include, but are not limited to, cartilage (including, elastic, hyaline, and fibrocartilage), collagen, adipose tissue, reticular connective tissue, embryonic connective tissues (including mesenchymal connective tissue and mucous connective tissue), tendons, ligaments, and bone.

The article of the present embodiments can also serve as or be part of other implants, including, without limitation, dental implants, mammary implants, penile implants.

When the article of the present embodiments serves for patching, it can be implanted over any organ in which collagen patches can be beneficial. Representative examples including, without limitation, arthroscopic patch, various types of hernia patches (e.g., inguinal hernia, femoral hernia, scrotal hernia, ventral hernia, umbilical hernia, ventral/epigastric hernia, incisional hernia, spigelian hernia, recurrent hernia, recurrent incisional hernia, bilateral hernia, stoma hernia, and hiatus hernia), visceral patch and the like.

When the article is used for coating or wrapping an organ, it can be adapted for wrapping or coating various types of organs, typically elongated organs, such as, but not limited to, bones, blood vessels (arteries, veins, etc), nerve (e.g., a peripheral nerve such as the median nerve of the wrist) and tendons. When nerve tissue is damaged, scar tissue may forms in and around the injury site, which not only affects nerve signal transmittance and axonal growth across the injury site, but also develops painful neuroma. Similarly, scar tissue formation caused by an injury to a tendon can result tendon adhesion to the surrounding tissue, which, if the tissue is in the joint region, can lead to immobilization of the joint. In addition, a tendon injury may also induce adhesion between the tendon and an adjacent nerve, resulting in severe pain and loss of productivity. The article of the present embodiments can be used for reducing or minimizing scar tissue formation within and around the injury site, by wrap around the injury site to prevent invasion of fibrogenic cells.

When the article is used for connecting two or more organs, it can be adapted for connecting two or more tendons, two or more ligaments, tendon to bone and the like.

The article of the present embodiments can also be used as, or be part of soft and hard tissue prostheses including, without limitation, pumps, electrical devices including stimulators and recorders, auditory prostheses and pacemakers.

The article of the present embodiments can also be shaped and be subsequently used for reconstructing an organ, such as, but not limited to, a breast, a face, or a body part after cancer surgery or trauma.

When the article is used as an artificial organ, it can be shaped according to the shape of the organ. The article of the present embodiments can be used as, or be a part in, an artificial organ selected from a group consisting of tissues or organs of the circulatory system, tissues or organs of the blood vessel system, tissues or organs of the digestive track, tissues or organs of the gut-associated glands, tissues or organs of the respiratory system, or tissues or organs of the urinary system, liver lobules and artificial alveolar structures. For example, the article of the present embodiments can be used as, or be a part in, an artificial organ selected from a group consisting of a biological patch, a vascular graft, a heart valve, a venous valve, a tendon, a craniofacial tendon, a ligament, a bone, a muscle, a cartilage, a ureter, a urinary bladder, a dermal graft, a cardiac tissue, an anti-adhesion membrane, a myocardial tissue, a lung, a pancreas, a larynx, a joint, a meniscus, and a disk.

The collagen of the present embodiments can comprise a sufficient portion of its telopeptides such that under suitable conditions it is capable of forming fibrils.

Thus, for example, the collagen may be atelocollagen, a telocollagen or procollagen.

As used herein, the term “atelocollagen” refers to collagen molecules lacking both the N- and C-terminal propeptides typically comprised in procollagen and at least a portion of its telopeptides, but including a sufficient portion of its telopeptides such that under suitable conditions it is capable of forming fibrils.

The term “procollagen” as used herein, refers to a collagen molecule (e.g. human) that comprises either an N-terminal propeptide, a C-terminal propeptide or both. Exemplary human procollagen amino acid sequences are set forth by SEQ ID NOs: 3, 4, 5 and 6.

The term “telocollagen” as used herein, refers to collagen molecules that lack both the N- and C-terminal propeptides typically comprised in procollagen but still contain the telopeptides. The telopeptides of fibrillar collagen are the remnants of the N- and C-terminal propeptides following digestion with native N/C proteinases.

According to another embodiment, the collagen is devoid of its telopeptides and is not capable of undergoing fibrillogenesis.

According to another embodiment, the collagen is a mixture of the types of collagen above.

The collagen may be isolated from an animal (e.g. bovine, pig or human) or may be genetically engineered using recombinant DNA technology.

Methods of isolating collagen from animals are known in the art. Dispersal and solubilization of native animal collagen can be achieved using various proteolytic enzymes (such as porcine mucosal pepsin, bromelain, chymopapain, chymotrypsin, collagenase, ficin, papain, peptidase, proteinase A, proteinase K, trypsin, microbial proteases, and, similar enzymes or combinations of such enzymes) which disrupt the intermolecular bonds and remove the immunogenic non-helical telopeptides without affecting the basic, rigid triple-helical structure which imparts the desired characteristics of collagen (see U.S. Pat. Nos. 3,934,852; 3,121,049; 3,131,130; 3,314,861; 3,530,037; 3,949,073; 4,233,360 and 4,488,911 for general methods for preparing purified soluble collagen). The resulting soluble collagen can be subsequently purified by repeated precipitation at low pH and high ionic strength, followed by washing and re-solublization at low pH.

Plants expressing collagen chains and procollagen are known in the art, see for example, WO06035442A3; Merle et al., FEBS Lett. 2002 Mar. 27; 515(1-3):114-8. PMID: 11943205; and Ruggiero et al., 2000, FEBS Lett. 2000 Mar. 3; 469(1):132-6. PMID: 10708770; and U.S. Pat. Applications 2002/098578 and 2002/0142391 as well as U.S. Pat. No. 6,617,431 each of which are incorporated herein by reference.

It will be appreciated that the present embodiments also contemplate genetically modified forms of collagen/atelocollagen—for example collagenase-resistant collagens and the like [Wu et al., Proc Natl. Acad Sci, Vol. 87, p. 5888-5892, 1990].

Recombinant procollagen or telocollagen may be expressed in any non-animal cell, including but not limited to plant cells and other eukaryotic cells such as yeast and fungus.

Plants in which the human procollagen or telocollagen may be produced (i.e. expressed) may be of lower (e.g. moss and algae) or higher (vascular) plant species, including tissues or isolated cells and extracts thereof (e.g. cell suspensions). Preferred plants are those which are capable of accumulating large amounts of collagen chains, collagen and/or the processing enzymes described herein below. Such plants may also be selected according to their resistance to stress conditions and the ease at which expressed components or assembled collagen can be extracted. Examples of plants in which human procollagen may be expressed include, but are not limited to tobacco, maize, alfalfa, rice, potato, soybean, tomato, wheat, barley, canola, carrot, lettuce and cotton.

Production of recombinant procollagen is typically effected by stable or transient transformation with an exogenous polynucleotide sequence encoding human procollagen.

Production of human telocollagen is typically effected by stable or transient transformation with an exogenous polynucleotide sequence encoding human procollagen and at least one exogenous polynucleotide sequence encoding the relevant protease.

The stability of the triple-helical structure of collagen requires the hydroxylation of prolines by the enzyme prolyl-4-hydroxylase (P4H) to form residues of hydroxyproline within the collagen chain. Although plants are capable of synthesizing hydroxyproline-containing proteins, the prolyl hydroxylase that is responsible for synthesis of hydroxyproline in plant cells exhibits relatively loose substrate sequence specificity as compared with mammalian P4H. Thus, production of collagen containing hydroxyproline only in the Y position of Gly-X-Y triplets requires co-expression of collagen and human or mammalian P4H genes [Olsen et al, Adv Drug Deliv Rev. 2003 Nov. 28; 55(12):1547-67].

Thus, according to one embodiment, the procollagen or telocollagen is expressed in a subcellular compartment of a plant that is devoid of endogenous P4H activity so as to avoid incorrect hydroxylation thereof. As is used herein, the phrase “subcellular compartment devoid of endogenous P4H activity” refers to any compartmentalized region of the cell which does not include plant P4H or an enzyme having plant-like P4H activity. According to one embodiment, the subcellular compartment is a vacuole.

Accumulation of the expressed procollagen in a subcellular compartment devoid of endogenous P4H activity can be effected via any one of several approaches.

For example, the expressed procollagen/telocollagen can include a signal sequence for targeting the expressed protein to a subcellular compartment such as the apoplast or an organelle (e.g. chloroplast). Examples of suitable signal sequences include the chloroplast transit peptide (included in Swiss-Prot entry P07689, amino acids 1-57) and the Mitochondrion transit peptide (included in Swiss-Prot entry

P46643, amino acids 1-28).

Alternatively, the sequence of the procollagen can be modified in a way which alters the cellular localization of the procollagen when expressed in plants.

The present embodiments contemplate genetically modified cells co-expressing both human procollagen and a P4H, capable of correctly hydroxylating the procollagen alpha chain(s) [i.e. hydroxylating only the proline (Y) position of the Gly-X-Y triplets]. P4H is an enzyme composed of two subunits, alpha and beta as set forth in Genbank Nos. P07237 and P13674. Both subunits are necessary to form an active enzyme, while the beta subunit also possesses a chaperon function.

The P4H expressed by the genetically modified cells of the present embodiments is preferably a human P4H. In addition, P4H mutants which exhibit enhanced substrate specificity, or P4H homologues can also be used. A suitable P4H homologue is exemplified by an Arabidopsis oxidoreductase identified by NCBI accession no: NP_{—}179363.

Since it is essential that P4H co-accumulates with the expressed procollagen chain, the coding sequence thereof is preferably modified accordingly (e.g. by addition or deletion of signal sequences).

In mammalian cells, collagen is also modified by Lysyl hydroxylase, galactosyltransferase and glucosyltransferase. These enzymes sequentially modify lysyl residues in specific positions to hydroxylysyl, galactosylhydroxylysyl and glucosylgalactosyl hydroxylysyl residues at specific positions. A single human enzyme, Lysyl hydroxylase 3 (LH3), as set forth in Genbank No. 060568, can catalyze all three consecutive modifying steps as seen in hydroxylysine-linked carbohydrate formation.

Thus, the genetically modified cells of the present embodiments may also express mammalian LH3.

The procollagen(s) and modifying enzymes described above can be expressed from a stably integrated or a transiently expressed nucleic acid construct which includes polynucleotide sequences encoding the procollagen alpha chains and/or modifying enzymes (e.g. P4H and LH3) positioned under the transcriptional control of functional promoters. Such a nucleic acid construct (which is also termed herein as an expression construct) can be configured for expression throughout the whole organism (e.g. plant, defined tissues or defined cells), and/or at defined developmental stages of the organism. Such a construct may also include selection markers (e.g. antibiotic resistance), enhancer elements and an origin of replication for bacterial replication.

There are various methods for introducing nucleic acid constructs into both monocotyledonous and dicotyledenous plants (Potrykus, I., Annu. Rev. Plant. Physiol., Plant. Mol. Biol. (1991) 42:205-225; Shimamoto et al., Nature (1989) 338:274-276). Such methods rely on either stable integration of the nucleic acid construct or a portion thereof into the genome of the plant, or on transient expression of the nucleic acid construct, in which case these sequences are not inherited by the plant's progeny.

In addition, several methods exist in which a nucleic acid construct can be directly introduced into the DNA of a DNA-containing organelle such as a chloroplast.

There are two principle methods of effecting stable genomic integration of exogenous sequences, such as those included within the nucleic acid constructs of the present embodiments, into plant genomes:

(i) Agrobacterium-mediated gene transfer: Klee et al. (1987) Annu. Rev. Plant Physiol. 38:467-486; Klee and Rogers in Cell Culture and Somatic Cell Genetics of Plants, Vol. 6, Molecular Biology of Plant Nuclear Genes, eds. Schell, J., and Vasil, L. K., Academic Publishers, San Diego, Calif. (1989) p. 2-25; Gatenby, in Plant Biotechnology, eds. Kung, S. and Arntzen, C. J., Butterworth Publishers, Boston, Mass. (1989) p. 93-112.

(ii) Direct DNA uptake: Paszkowski et al., in Cell Culture and Somatic Cell Genetics of Plants, Vol. 6, Molecular Biology of Plant Nuclear Genes eds. Schell, J., and Vasil, L. K., Academic Publishers, San Diego, Calif. (1989) p. 52-68; including methods for direct uptake of DNA into protoplasts, Toriyama, K. et al. (1988) Bio/Technology 6:1072-1074. DNA uptake induced by brief electric shock of plant cells: Zhang et al. Plant Cell Rep. (1988) 7:379-384. Fromm et al. Nature (1986) 319:791-793. DNA injection into plant cells or tissues by particle bombardment, Klein et al. Bio/Technology (1988) 6:559-563; McCabe et al. Bio/Technology (1988) 6:923-926; Sanford, Physiol. Plant. (1990) 79:206-209; by the use of micropipette systems: Neuhaus et al., Theor. Appl. Genet. (1987) 75:30-36; Neuhaus and Spangenberg, Physiol. Plant. (1990) 79:213-217; or by the direct incubation of DNA with germinating pollen, DeWet et al. in Experimental Manipulation of Ovule Tissue, eds. Chapman, G. P. and Mantell, S. H. and Daniels, W. Longman, London, (1985) p. 197-209; and Ohta, Proc. Natl. Acad. Sci. USA (1986) 83:715-719.

There are various methods of direct DNA transfer into plant cells. In electroporation, protoplasts are briefly exposed to a strong electric field. In microinjection, the DNA is mechanically injected directly into the cells using very small micropipettes. In microparticle bombardment, the DNA is adsorbed on microprojectiles such as magnesium sulfate crystals, tungsten particles or gold particles, and the microprojectiles are physically accelerated into cells or plant tissues.

Regardless of the transformation technique employed, once procollagen-expressing progeny are identified, such plants are further cultivated under conditions which maximize expression thereof. Progeny resulting from transformed plants can be selected, by verifying presence of exogenous mRNA and/or polypeptides by using nucleic acid or protein probes (e.g. antibodies). The latter approach enables localization of the expressed polypeptide components (by for example, probing fractionated plants extracts) and thus also verifies the plant's potential for correct processing and assembly of the foreign protein.

Following cultivation of such plants, the telopeptide-comprising collagen is typically harvested. Plant tissues/cells are preferably harvested at maturity, and the procollagen molecules are isolated using extraction approaches. Preferably, the harvesting is effected such that the procollagen remains in a state that it can be cleaved by protease enzymes. According to one embodiment, a crude extract is generated from the transgenic plants of the present embodiments and subsequently contacted with the protease enzymes.

As mentioned, the propeptide or telopeptide-comprising collagen may be incubated with a protease to generate atelocollagen or collagen prior to preparation of mesophase solutions. It will be appreciated that the propeptide or telopeptide-comprising collagen may be purified from the genetically engineered cells prior to incubation with protease, or alternatively may be purified following incubation with the protease. Still alternatively, the propeptide or telopeptide-comprising collagen may be partially purified prior to protease treatment and then fully purified following protease treatment. Yet alternatively, the propeptide or telopeptide-comprising collagen may be treated with protease concomitant with other extraction/purification procedures.

Exemplary methods of purifying or semi-purifying the telopeptide-comprising collagen of the present embodiments include, but are not limited to salting out with ammonium sulfate or the like and/or removal of small molecules by ultrafiltration.

According to one embodiment, the protease used for cleaving the recombinant propeptide or telopeptide comprising collagen is not derived from an animal. Exemplary proteases include, but are not limited to certain plant derived proteases e.g. ficin (EC 3.4.22.3) and certain bacterial derived proteases e.g. subtilisin (EC 3.4.21.62), neutrase. The present inventors also contemplate the use of recombinant enzymes such as rhTrypsin and rhPepsin. Several such enzymes are commercially available e.g. Ficin from Fig tree latex (Sigma, catalog #F4125 and Europe Biochem), Subtilisin from Bacillus licheniformis (Sigma, catalog #P5459) Neutrase from bacterium Bacillus amyloliquefaciens (Novozymes, catalog #PW201041) and TrypZean™, a recombinant human trypsin expressed in corn (Sigma catalog #T3449).

As used herein, the phrase “collagen fiber” refers to a non-soluble self-aggregate of collagen comprising a fibrous structure in which collagen molecules are packed in series and also in parallel. It will be appreciated that the collagen molecules may be in their monomeric form or their polymeric form. The collagen fibers generated according to the method of the present embodiments typically have a cross sectional diameter in the range of about 2 microns to 70 microns and more preferably between 5 microns and 30 microns.

As mentioned, the starting material for generating the collagen structure of the present embodiments is collagen (or procollagen) in a liquid crystal form.

Liquid crystal is a state of matter that is intermediate between the crystalline solid and the amorphous liquid. There are three basic phases of liquid crystals, known as smectic phase, nematic phase, and cholesteric phase and the present embodiments envisage the use of any of the above. In the smectic phase a one-dimensional translational order, as well as orientational order exists. In the nematic phase, only a long-range orientational order of the molecular axes exists. Cholesteric phase is also a nematic liquid type with molecular aggregates lie parallel to one another in each plane, but each plane is rotated by a constant angle from the next plane.

According to one embodiment, the liquid collagen solution is an acidic solution of collagen monomers (e.g. human or bovine collagen type I). Exemplary acids for solubilizing monomeric collagen include, but are not limited to hydrochloric acid (HCl) and acetic acid.

As used herein, the phrase “collagen monomers” refers to monomeric collagen that has not undergone the process of polymerization.

According to one embodiment a concentration of about 1 mM-100 mM HCl is used to solubilize the collagen monomers. An exemplary concentration of HCl which may be used to solubilize collagen monomers is about 10 mM HCl.

According to one embodiment a concentration of about 0.05 mM-50 mM acetic acid is used to solubilize the collagen monomers. An exemplary concentration of acetic acid which may be used to solubilize collagen monomers is about 0.5 M acetic acid.

The present embodiments contemplate addition of a crosslinker to the acidic solution of collagen monomers. The acidity of the solution prevents premature crosslinking. Following extrusion into a neutral coagulating solution, the crosslinker becomes activated and crosslinks the collagen fibrils. Examples of crosslinkers are further described herein below.

It will be appreciated that once the collagen is solubilized in the acid, the pH of the solution may be increased. The pH is selected such that the collagen therein still displays liquid crystal properties. Raising of the pH may be effected by dialyzing the acidic collagen against a higher pH buffer (e.g. pH 4/4.5 acetate buffer).

The present inventors have shown that when such a solution is extruded into a low phosphate buffer, this dope did not dissolve, and coagulated into a white, opaque fiber. The fiber maintained its shape and swelled substantially less then acidic dope fibers.

Generating solutions of liquid crystalline collagen monomers may be effected by concentrating a liquid collagen solution. The liquid collagen solution may be concentrated using any means known in the art, including but not limited to filtration, rotary evaporation and dialysis membrane.

Dialysis may be effected against a hygroscopic polymer, for example, PEG, a polyethylene oxide, amylose or sericin. Preferably, the PEG is of a molecular weight of 10,000-30,000 g/mol and has a concentration of 25-50%. According to a particular embodiment, a slide-a-lyzer dialysis cassette (Pierce, MW CO 3500) is used. Typically, the dialysis is effected in the cold (e.g. at about 4° C.). The dialysis is effected for a time period sufficient to result in a final concentration of aqueous collagen solution of about 10 mg/ml or more. According to one embodiment, the solution of monomeric collagen is at a concentration of about 100-200 mg/ml or between 0.7-0.3 mM.

In most cases dialysis for 2-16 hours is sufficient, depending on volume and concentration.

According to another embodiment, the solution of liquid crystalline collagen comprises high concentrations (5-30 mg/ml, depending on the collagen type) of procollagen molecules in physiological buffer. It has been shown that such solutions develop long range nematic and precholesteric liquid crystal ordering extending over 100 μm² domains, while remaining in solution (R. Martin et al., J. Mol. Biol. 301: 11-17 (2000)). Procollagen concentrations in vivo are estimated at several tens of milligrams per milliliter in the secretory vesicles and the molecules are often observed to be aligned in a nematic-like ordering.

In another embodiment, the starting collagen material may be prepared by ultrasonic treatment. Brown E. M. et al. Journal of American Leather Chemists Association, 101:274-283 (2006), herein incorporated by reference by its entirety.

The solutions of liquid crystalline collagen may comprise additives such as ATP to decrease the threshold of the required concentration to develop the liquid crystal state. Without being bound by any particular theory, generally, highly negative charged molecules (more that −3) can be used as additives to the collagen solution to promote the orientation or adhesion of the collagen, so that the collagen can form liquid crystals at relatively lower concentration. Suitable additives include, but are not limited to ATP, vanadate, insulin, phosphate and VGF.

Other additives that may be added to the starting material of the present embodiments include antimicrobials such as silver nitrate, iodized radicals (e.g., Triosyn®; Hydro Biotech), benzylalkonium chloride, alkylpyridinium bromide (cetrimide), and alkyltrimethylammonium bromide. Viscosity enhancers may be added to improve the rheological properties of the starting material. Examples include, but are not limited to polyacrylates, alginate, cellulosics, guar, starches and derivatives of these polymers, including hydrophobically modified derivatives.

The present embodiments further contemplates addition of hyaluronic acid (HA) to the solution.

When a coagulating solution is employed, it serves to stabilize or preserve the molecular orientation of the extruded collagen molecules. Typically, the stabilizing agent in the coagulating solution is at a high enough osmolarity such that is can extract water from the collagen mesophase and dry it.

The coagulating solution can comprise an organic solvent. The present embodiments contemplate coagulating solutions wherein at least 50% thereof comprises the organic solvent. The present embodiments further contemplates coagulating solutions wherein at least 70% thereof comprises the organic solvent. The present embodiments further contemplate coagulating solutions wherein at least 90% thereof comprises the organic solvent.

The collagen can remain in the coagulating solution for at least 15 minutes.

Exemplary organic solvents that may be used according the present embodiments include, but are not limited to acetone, methanol, isopropanol, methylated spirit and ethanol.

Alternatively, the coagulating solution may be a concentrated aqueous salt solution having a high ionic strength. The high osmotic pressure of a concentrated salt solution draws the water away from the collagen protein, thereby facilitating fiber coagulation. Preferred coagulating solutions include aqueous solutions containing a high concentration of aluminum sulfate, ammonium sulfate, sodium sulfate, or magnesium sulfate. Additives, particularly acids, such as acetic acid, sulfuric acid, or phosphoric acid, or also sodium hydroxide may be added to the salt-based coagulation bath.

Contemplated salt coagulating solutions may comprise one or more salts of high solubility such as, for example, salts containing one or more of the following anions: nitrates, acetates, chlorates, halides (fluoride, chloride, bromide, iodide), sulfates, sulfides, sulfites, carbonates, phosphates, hydroxides, thiocyanates, bicarbonates, formates, propionates, and citrates; and one or more of the following cations: ammonium, aluminum, calcium, cesium, potassium, lithium, magnesium, manganese, sodium, nickel, rubidium, antimony, and zinc. The solution may also contain an acid of the same anion as the salt, e.g., nitric, acetic, hydrochloric, sulfuric, carbonic, phosphoric, formic, propionic, citric, or lactic acid, or another acid which also forms highly soluble salts with the cation(s) present. Preferably, the salts used in the coagulating solution of the present embodiments are multivalent anions and/or cations, resulting in a greater number of ions, and proportionally higher ionic strength, on dissociation. Typically, concentrated salt coagulating solutions comprise about 30%-70% (w/v) of salt; preferably about 40-65%.

According to another embodiment the coagulation solution is a solution that allows polymerization (i.e. fibrilogenesis) of collagen monomers. Such a solution typically is at a neutral or high pH (e.g. pH 7.4 or more) to allow for polymerization. An exemplary fibrilogenesis buffer comprises between about 5 mM sodium phosphate to about 50 mM sodium phosphate.

Useful additives may be included in the coagulating medium include, but are not limited to surfactants, osmoprotective agents, stabilizing agents, UV inhibitors, and antimicrobial agents. Stabilizers that protect against UV radiation, radical formation, and biodegradation include, for example, 2-hydroxybenzophenones, 2-hydroxyphenyl-2-(2H)-benzotriazoles, cifmamates, and mixtures thereof. These chemicals are capable of absorbing and dissipating UV energy, thereby inhibiting UV degradation. Free radicals are neutralized by hindered amine light stabilizers (HALS), butylated hydroxyanisole (BHA), and butylated hydroxytoluene (BHT).

The collagen of the present embodiments can be crosslinked using any one of the below methods: 1. by glutaraldehyde and other chemical crosslinking agents; 2. by glycation using different sugars; 3. by Fenton reaction using metal ions such as copper; 4. by lysine oxidase; or 5. by UV radiation.

The collagen structures generated according to the method of the present embodiments may be used per se, or as part of a composite material. The components of the composites of the present embodiments may be attached to, coated on, embedded or impregnated into the collagen of the present embodiments. In such composites, the collagen may be uncrosslinked, partially crosslinked or fully crosslinked. Exemplary components of the composite material include, but are not limited to minerals, pharmaceutical agents (i.e. drugs) polysaccharides and polypeptides.

Exemplary polysaccharides that may be used in composite materials of the present embodiments include, but are not limited to glycosaminoglycans such as chondroitin sulfate of type A, C, D, or E, dermatan sulfate, keratan sulfate, heparan sulfate, heparin, hyaluronic acid and their derivatives, individually or mixed.

Exemplary polypeptides that may be used in composite materials of the present embodiments include, but are not limited to resilin, silk, elastin and fibronectin.

Exemplary minerals that may be used in composite materials of the present embodiments include, but are not limited to calcium, magnesium, boron, zinc, copper, manganese, iron, silicon, selenium, phosphorus and sulfur. Methods for preparing collagen mineral composites are well known in the art, see for example WO/2006/118803.

Therapeutic compounds or agents that modify cellular activity can also be incorporated (e.g. attached to, coated on, embedded or impregnated) into the collagen structure or a portion thereof. In addition, agents that act to increase cell attachment, cell spreading, cell proliferation, cell differentiation and/or cell migration in the collagen structure may also be incorporated into the collagen structure. Such agents can be biological agents such as an amino acid, peptides, polypeptides, proteins, DNA, RNA, lipids and/or proteoglycans.

Suitable proteins which can be used along with the present embodiments include, but are not limited to, extracellular matrix proteins [e.g., fibrinogen, collagen, fibronectin, vimentin, microtubule-associated protein 1D, Neurite outgrowth factor (NOF), bacterial cellulose (BC), laminin and gelatin], cell adhesion proteins [e.g., integrin, proteoglycan, glycosaminoglycan, laminin, intercellular adhesion molecule (ICAM) 1, N-CAM, cadherin, tenascin, gicerin, RGD peptide and nerve injury induced protein 2 (ninjurin2)], growth factors [epidermal growth factor, transforming growth factor-α, fibroblast growth factor-acidic, bone morphogenic protein, fibroblast growth factor-basic, erythropoietin, thrombopoietin, hepatocyte growth factor, insulin-like growth factor-I, insulin-like growth factor-II, Interferon-β, platelet-derived growth factor, Vascular Endothelial Growth Factor and angiopeptin], cytokines [e.g., M-CSF, IL-1beta, IL-8, beta-thromboglobulin, EMAP-II, G-CSF and IL-10], proteases [pepsin, low specificity chymotrypsin, high specificity chymotrypsin, trypsin, carboxypeptidases, aminopeptidases, proline-endopeptidase, Staphylococcus aureus V8 protease, Proteinase K (PK), aspartic protease, serine proteases, metalloproteases, ADAMTS17, tryptase-gamma, and matriptase-2] and protease substrates.

Additionally and/or alternatively, the collagen structures of the present embodiments may comprise an antiproliferative agent (e.g., rapamycin, paclitaxel, tranilast, Atorvastatin and trapidil), an immunosuppressant drug (e.g., sirolimus, tacrolimus and Cyclosporine) and/or a non-thrombogenic or anti-adhesive substance (e.g., tissue plasminogen activator, reteplase, TNK-tPA, glycoprotein IIb/IIIa inhibitors, clopidogrel, aspirin, heparin and low molecular weight heparins such as enoxiparin and dalteparin).

As used herein the term “about” refers to ±10%.

The word “exemplary” is used herein to mean “serving as an example, instance or illustration.” Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments.” Any particular embodiment of the invention may include a plurality of “optional” features unless such features conflict.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### Example 1

### Spirally Shaped Collagen Structure

A layered structure with two layers was fabricated according to the teachings of some embodiments of the present invention. The structure included a layer A and a layer B. Layer A included 0.5% GTA. Once applied, layer A was air dried in order for the collagen monomers to arrange themselves in the order of the shear applied. Thereafter, layer B, which included 0.1% GTA, was applied onto layer A, and in the same direction. Layer B was also air dried similarly to layer A.

After drying, the structure was crosslinked and fibrilized simultaneously by immersion in a fibrilogenesis buffer containing a buffering agent, salt and 0.1% GTA. The structure was incubated in the buffer at 37° C. for about 2 days.

The structure was then treated in water at a temperature of about 90° C. to induce shrinkage, resulting in a spirally shaped structure.

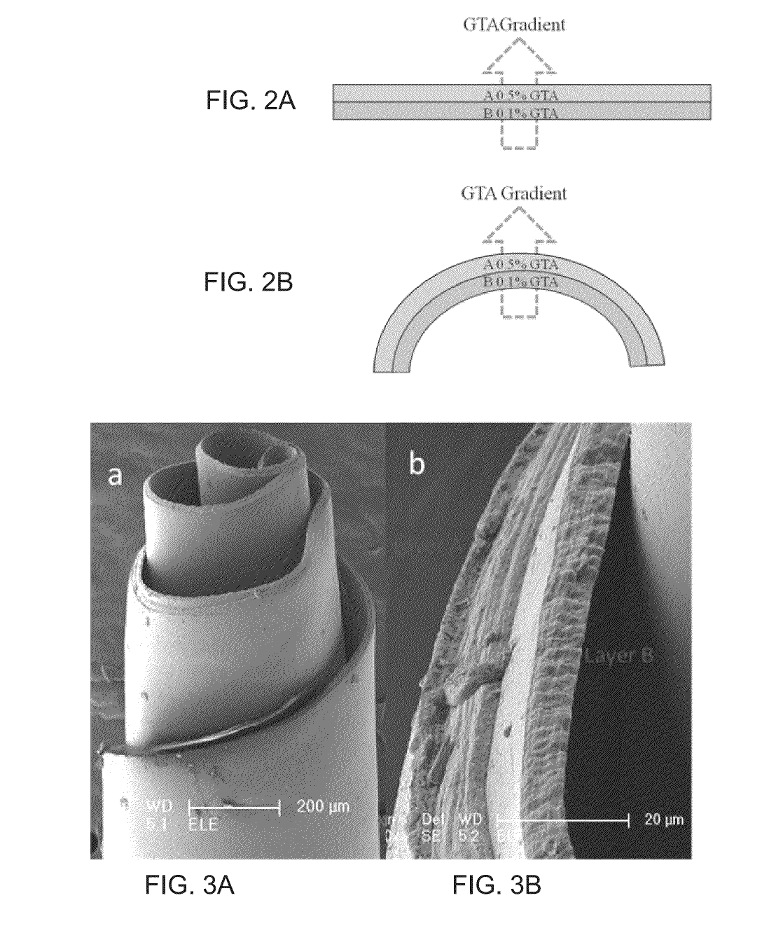

FIG. 2A shows the article after incubation in the buffer, and FIG. 2B shows the article after the shrinkage.

FIG. 3A-B are Scanning Electron Microscopy (SEM) images of the resulting structure. FIG. 3A shows the spirally shaped structure. FIG. 3B shows the structure in a larger magnification. A difference in texture was observed between the layers.

### Example 2

### A Hollow Collagen Structure

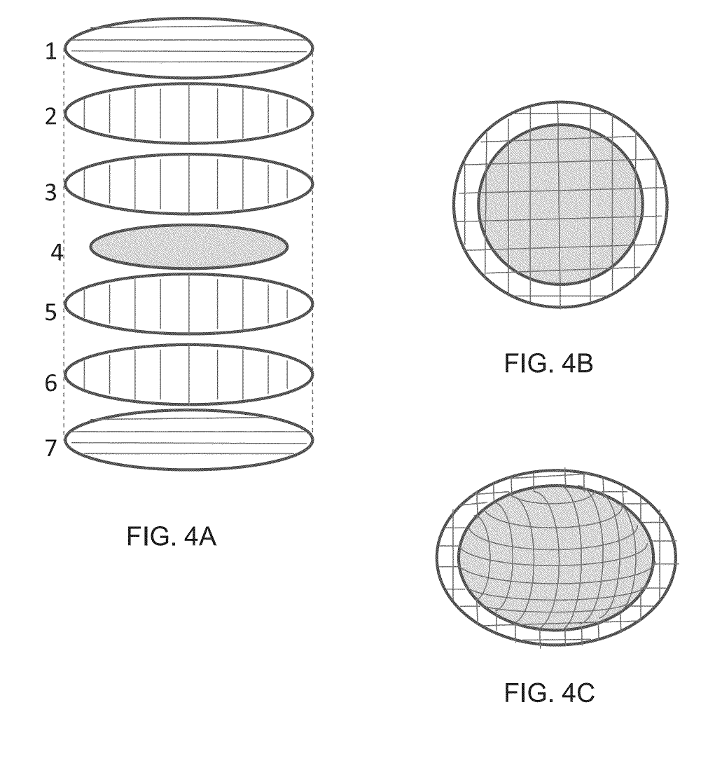

FIGS. 4A-C are schematic illustration of a procedure for fabricating a hollow collagen structure with a void between two layers, according to some embodiments of the present invention. Such a structure can be used, for example, as a bladder or the like. In the present example, which is not to be construed as limiting, the layered structure includes 7 layers.

FIG. 4A is an exploded view of the layered structure. Layer 1 is a support layer with fiber direction as indicated. GTA concentration is set to create maximum crosslinking (e.g., 0.5%). Layers 2 and 3 are (fibrillarly) directed perpendicularly to layer 1. A descending GTA gradient is established from layer 2 to 3, so as to create the desired curvature between them after thermal treatment. Layer 4 is a spacer layer which serves for avoiding crosslinking and/or attachment between layers 3 and 4. Layers 5 and 6 are the same as layers 3 and 2, respectively. Thus, the direction of the GTA gradient from layer 5 to layer 6 is opposite to the direction of the GTA gradient from layer v2 to layer 3 (i.e., descending GTA gradient from layer 6 to 5). Layer 7 is another support layer which can be the same as layer 1.

FIGS. 4B-C are schematic illustrations of a top view of the layered structure before (FIG. 4B) and after (FIG. 4C) thermal treatment. As illustrated, a hollow balloon is obtained.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method of fabricating a collagen structure, comprising applying onto a surface in a layerwise manner acidic solutions of liquid crystalline collagen and a crosslinker to form a layered structure, wherein acidic solutions corresponding to at least two adjacent layers have different concentrations of said crosslinker therein.

2. The method of claim 1, wherein said surface is a solid surface.

3. The method of claim 1, wherein said surface is a surface of a liquid.

4. The method of claim 1, wherein said surface is a surface of a gel.

5. The method according to claim 1, wherein at least one of said acidic solutions has a pH of from about 2 to about 5.

6. The method according to claim 1, further comprising at least partially drying each layer prior to the application of a subsequent layer.

7. The method according to claim 1, further comprising drying said layered structure collectively.

8. The method according to claim 1, wherein said applying said acidic solutions for forming said at least two of layers is executed generally along the same direction.

9. The method according to claim 1, wherein said applying said acidic solutions is executed generally along the same direction for all layers.

10. The method according to claim 1, further comprising activating said crosslinker.

11. The method according to claim 10, further comprising fibrilizing said collagen.

12. An article, comprising a plurality of layers each being made of a non-fibrilized collagen material and being incorporated with an activatable crosslinker which is in an inactive form, said layers being arranged to form a layered structure wherein at least two adjacent layers in said layered structure have different concentrations of said crosslinker therein.

13. A method of fabricating a collagen structure, comprising activating the inactive crosslinker in the layered structure of the article of claim 12.

14. The method according to claim 13, further comprising fibrilizing said collagen material.

15. The method according to claim 11, wherein said fibrillization and said activation are executed contemporaneously.

16. The method according to claim 10, wherein said fibrillization comprises immersing the article in a coagulation medium.

17. The method according to claim 1, wherein said crosslinker comprises a material selected from the group consisting of Glutaraldehyde, DOPA, Tyramine and Methacryl.

18. An article, comprising a plurality of layers each being made of a fibrilized and crosslinked collagen material, said layers being arranged to form a layered structure, wherein at least one layer in said layered structure is characterized by a level of crosslinking which is different from a level of crosslinking characterizing a layer being adjacent thereto.

19. A method of fabricating a collagen structure, comprising applying a thermal treatment to the article of claim 18.

20. An article, comprising a plurality of layers each being made of a fibrilized and crosslinked collagen material, said layers being arranged to form a layered structure,
wherein at least one layer in said layered structure is non-planar and being characterized by a level of crosslinking which is different from a level of crosslinking characterizing a layer being adjacent thereto.

21. The article of claim 20, wherein a characteristic direction of alignment of said collagen material in said at least one layer is generally the same as a characteristic direction of alignment of said collagen material in said adjacent layer.

22-23. (canceled)

24. A method of treating a damaged organ in a subject in need thereof, comprising implanting the article of claim 20 into the subject, thereby treating the damaged organ.

25-28. (canceled)

29. The method according to claim 24, wherein said organ is selected from the group consisting of a bladder, a tendon, a bone, a brain, a cartilage, an esophagus, a fallopian tube, a heart valve, a pancreas, an intestine, a gall bladder, a kidney, a liver or liver lobule, a lung, a skeletal muscle, a skin, a spleen, a stomach, a thymus, a thyroid, a trachea, a ureter, a urethra, a urogenital tract, a uterus, a blood vessel and a cornea.

30-55. (canceled)
